(19) United States
(12) Patent Application Publication
(10) Pub. No.: US 2007/0172496 A1
(43) Pub. Date: Jul. 26, 2007

(54) Conjugates of amyloid proteins as vaccines for amyloid-related diseases

(75) Inventor: Ole Frilev Olesen, Valby (DK)

Correspondence Address: THORPE NORTH & WESTERN, LLP., 8180 SOUTH 700 EAST, SUITE 200, SANDY, UT, 84070, US

(73) Assignee: Curix ApS, Valby (DK)

(21) Appl. No.: 10/587,816

(22) PCT Filed: Jan. 28, 2005
(86) PCT No.: PCT/DK05/00067

§ 371 Date: Mar. 28, 2007

(30) Foreign Application Priority Data
Jan. 28, 2004 (DK) PA 2004 00117

(51) Int. Cl. A61K 39/08; C07K 14/33

(52) U.S. Cl. 424/239.1; 530/350

(57) ABSTRACT

New conjugates comprising fragments of amyloid proteins, which may be used in vaccines for the treatment, prevention and/or amelioration of diseases associated with deposition of amyloid proteins, such as, e.g. Alzheimers disease. Methods for treating, preventing and/or ameliorating amyloid-related diseases, by administering a conjugate comprising fragments of an amyloid protein to a subject in need thereof, thereby enabling the production of antibodies in the subject. Antibodies—being capable of interacting with pathological regions within an amyloid protein, and thereby preventing e.g. the formation of amyloid fibrils, plaques and/or deposits, and methods for passive immunization wherein an antibody as described above is administered to a subject in need thereof. Furthermore is provided specific fragments of the C-terminal part of amyloid beta (1-42), that when administered to a mammal generates antibodies, which specifically targets the soluble form of the highly amyloidogenic amyloid beta (1-42).

### FIELD OF THE INVENTION

The present invention relates to new conjugates comprising fragments of amyloid proteins. The conjugates may be used in vaccines for the treatment, prevention and/or amelioration of diseases associated with deposition of amyloid proteins, such as, e.g. Alzheimer's disease. The invention also provides methods for treating, preventing and/or ameliorating amyloid-related diseases, by administering a conjugate comprising fragments of an amyloid protein to a subject in need thereof, thereby enabling the production of antibodies in the subject. The invention also encompasses antibodies being capable of interacting with pathological regions within an amyloid protein, and thereby preventing e.g. the formation of amyloid fibrils, plaques and/or deposits, and methods for passive immunization wherein an antibody as described above is administered to a subject in need thereof.

### BACKGROUND OF THE INVENTION

Amyloid diseases or amyloidoses include a number of disease states having a wide variety of outward symptoms. These disorders have in common the presence of abnormal extracellular deposits of protein fibrils, known as “amyloid fibrils”, “amyloid deposits” or “amyloid plaques” that are usually about 10-100 nm in diameter and are localized to specific organs or tissue regions. Such plaques are composed primarily of a naturally occurring soluble protein or peptide. These insoluble deposits are composed of generally lateral aggregates of fibrils that are approximately 10-15 nm in diameter. Though diverse in their occurrence, all amyloid deposits have common morphologic properties, stain with specific dyes (e.g. Thioflavin T, Congo red), and have a characteristic red-green birefringent appearance in polarized light after staining.

Amyloid-related diseases are characterised by the type of protein present in the deposit. For example, neurodegenerative diseases such as scrapie, bovine spongiform encephalitis, Creutzfeldt-Jakob disease and the like are characterized by the appearance and accumulation of a protease-resistant form of a prion protein (referred to as AScr or PrP-27) in the central nervous system. Similarly, Alzheimer's disease, another neurodegenerative disorder, is characterized by the deposition of amyloid plaques and neurofibrillary tangles. In this case, the plaque and blood vessel amyloid is formed by the deposition of fibrillar amyloid beta protein. Other diseases such as adult-onset diabetes (Type II diabetes) are characterized by the localized accumulation of amyloid in the pancreas.

Each amyloidogenic protein has the ability to fold into beta-sheets and to form insoluble fibrils, which get deposited extracellularly or intracellularly. Each amyloidogenic protein, although different in amino acid sequence, has the same property of forming fibrils and binding to other elements such as proteoglycan, amyloid P and complement component. Moreover, each amyloidogenic protein has amino acid sequences, which, although different, can catalyze the formation of beta-sheet structures.

In specific cases, amyloidogenic proteins, proto-fibrils and amyloidotic fibrils can be toxic to the surrounding cells. As per example, the amyloid beta fibrils have been associated with dead neuronal cells and microgliosis in patients with Alzheimer's disease. When tested in vitro, the amyloid beta peptide was shown to be capable of triggering an activation process of microglia (brain macrophages), which would explain the presence of microgliosis and brain inflammation found in the brain of patients with Alzheimer's disease.

In another type of amyloidosis seen in patients with Type II diabetes, the amyloidogenic protein IAPP has been shown to induce beta-islet cell toxicity in vitro. Hence, appearance of IAPP fibrils in the pancreas of Type II diabetic patients could contribute to the loss of the beta islet cells (Langerhans) and organ dysfunction.

One of the most prominent amyloid diseases is Alzheimer's disease, which is a progressive neurodegenerative disease affecting approximately 0.5-1% of the total population in the western world. Alzheimer's disease is characterized by the deposition of large numbers of amyloid plaques in the brain. This deposition is assumed to cause the pathology of the disease and most approaches to prevent Alzheimer's disease is aimed at reducing, removing, or preventing the formation of amyloid plaques. The main constituent of the amyloid plaques is the amyloid beta peptide (Aβ), a 40-42 amino-acid protein that is produced through cleavage of the amyloid precursor protein (APP).

It has been shown that an immunological response towards Aβ (1-42) peptides can reduce the deposition of Aβ in transgenic murine models of Alzheimer's disease (denoted AD). Schenk et al. (Nature, 1999) provided the first evidence by demonstrating that immunization Aβ (1-42) can diminish or prevent the accumulation of Aβ deposits in the brain. Parenteral immunization with aggregated synthetic Aβ (1-42) in Freund's adjuvant significantly decreased the number and density of Aβ deposits in the brain, with concomitant improvements in neuritic dystrophy and gliosis. Morgan et al (Nature, 2000) extended this line of evidence by demonstrating that immunization with Aβ (1-42) prevents memory loss in transgenic mice. Several studies have since confirmed these observations in different transgenic mouse models using various immunization strategies, including nasal vaccination (Weiner, et. al. Ann Neurol. 2000). The underlying biological mechanism is not clear yet, but the formation of anti-Aβ antibodies upon immunization clearly plays a primary role. Passive transfer of antibodies against Aβ can thus reduce the deposition of Aβ in a similar manner as active immunization in transgenic mouse models (Bard et al. Nature Med., 2000).

This has resulted in two broad hypotheses that are not necessarily mutually exclusive. The first hypothesis proposes an Fc-mediated uptake and clearance of Aβ-antibody complexes by activated microglia in the brain, which would require some diffusion of serum anti-Aβ antibodies across the blood-brain barrier. The second hypothesis suggests that peripheral antibodies reduce the plasma levels of Aβ, and thereby changes the equilibrium of Aβ between plasma and CNS. This results in net movement of Aβ out of the brain, and the plasma antibodies thus act as a peripheral “sink” for Aβ.

The extraordinary promising results from murine models resulted in a rapid translation into a human vaccine, AN1792, consisting of synthetic pre-aggregated Aβ (1-42) emulsified in the adjuvant QS21. A small phase I study revealed no safety concerns by parenteral administration of the human vaccine, and the clinical evaluation was therefore continued in a phase IIa trial in patients with mild to moderate AD. In this study, patients developing effective levels of Aβ antibodies showed slower rates of cognitive decline than controls (Hock et al., Neuron, 2003).

However, the clinical testing of AN1792 was discontinued prematurely when roughly 6% of patients developed meningoencephalitis. None of the rodents or other animals exposed to the vaccine during preclinical testing had indicated this problem, which can be explained by different observations. Firstly, animals were boosted with the vaccine using incomplete Freunds adjuvant (IFA), while the human vaccine was emulsified in QS21. IFA induces an immune response of the Th2 type, whereas QS21 induces a predominantly Th1 response. Secondly, full-length, aggregated Aβ molecules as used in AN1792 can be neurotoxic and contain several immunological features, including a complement activating sequence and may thus elicit the development of an acute inflammatory response. Full length Aβ, even in antigenic amounts, may therefore elicit or fuel non-specific immunological reactions. Thirdly, the transgenic mice have an elevated production of endogenous Aβ, which may cause T cell tolerance and therefore a decreased response to immunizations. Vaccination with full length Aβ may therefore induce a weaker immune response in transgenic animals than in native humans.

Using an endogenous protein as a vaccine (or a protein naturally present in the animal being vaccinated) such as in AN1792 is also associated with other drawbacks, including the possible development of autoimmune disease due to the generation of antibodies against “self” protein, and difficulty in eliciting an immune response due to the failure of the host immune system to recognize “self” antigens.

It has long been known that no direct correlation exist between neurological deficit and amyloid deposit burden. The lack of correlation is both evident in human AD patients, and in transgenic mice models of AD. Several lines of evidence also indicate that immobilized amyloid plaques are relatively harmless structures, whereas soluble Aβ oligomers are responsible for most of the neurotoxic and inflammatory damage associated with Aβ (Walsh, Nature 2000, Gong et al. PNAS USA 2003). Soluble Aβ oligomers have also been implicated in the physical degeneration of synapses (Mucke, Neurosci. 2000). The earlier attempts to prevent or treat AD through active vaccination approaches have resulted in immune responses towards both soluble and immobilised Aβ. These attempts have therefore been flawed by a relatively unspecific immune response that may actually have resulted in higher levels of soluble, toxic and potentially inflammatory Aβ oligomers. In the present invention, new conjugates and methods are devised that can overcome the existing hurdles by raising a strong and highly specific immune response towards the pathological forms of amyloid proteins.

### SUMMARY OF THE INVENTION

The present invention provides new conjugates for use in an active and/or passive vaccination strategy for the treatment, prevention and/or amelioration of an amyloid-related disease. The conjugate comprises P peptides (full-length or fragments of amyloid proteins) linked via the N-terminal end to a Ligand Presenting Assembly (LPA) as defined below, so that the P peptides will be C-terminally presented.

A conjugate according to the invention may have the following structure (I): 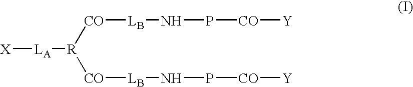
wherein
R represents the LPA backbone chosen from —N(CH₂—)₂, —NHCH< or —NHCH(CH₂—)₂,
X represents a hydrogen or a peptidic group, and
L_{A} is optionally present and may be an amino acid or a peptide containing at least 2 amino acid residues,
L_{B} is optionally present and may be an amino acid or a peptide containing at least 2 amino acid residues,
P is a peptide selected from full length or fragments of amyloid proteins or proteins with substantial similarity to an amyloid protein, and
Y is OH or NH₂,
   and pharmaceutically acceptable salts thereof.

The invention also relates to methods for treating, preventing and/or ameliorating amyloid-related diseases in mammals, by administering a conjugate comprising fragments of an amyloid protein to a subject in need thereof, thereby eliciting the production of antibodies in the subject and inducing an immune response in the mammal, thereby preventing or reducing amyloid-induced cellular toxicity and/or the formation of fibrils, plaques and/or amyloid deposits.

The invention also relates to antibodies being capable of interacting with pathological regions within an amyloid protein, and thereby preventing e.g. the formation of amyloid fibrils, plaques and/or deposits, and methods for passive immunization wherein an antibody as described above is administered to a subject in need thereof.

Furthermore, the present inventors have identified very specific fragments of the C-terminal part of amyloid beta (1-42), that when administered to a mammal generates antibodies, which specifically targets the soluble form of the highly amyloidogenic amyloid beta (1-42).

### DESCRIPTION OF THE INVENTION

The invention aims at developing a conjugate, which upon administration to a mammal is capable of eliciting a production of antibodies having specificity towards the conjugate itself and inducing an immune response in the mammal, thereby preventing or reducing amyloid-induced cellular toxicity and/or the formation of amyloid fibrils, plaques and/or deposits. More specifically, the antibodies produced should be specific towards one or more C-terminally presented P peptides of the conjugate. The general idea is, that the antibodies raised and having specificity against the C-terminally presented P peptides, are also capable of interacting with pathological regions within an amyloid protein, and thereby preventing e.g. the formation of amyloid fibrils, plaques and/or deposits, associated cellular toxicity and neurodegeneration.

Before going into details with the individual steps of the invention, in the following is given a list of specific terms used in the present text.

Definitions

The term “amyloid protein” or “amyloidogenic protein” is intended to denote a protein which is involved in the formation of fibrils, plaques and/or amyloid deposits, either by being part of the fibrils, plaques and/or deposits as such or by being part of the biosynthetic pathway leading to the formation of the fibrils, plaques and/or amyloid deposits. An amyloid protein can be derived from precursor proteins known to be associated with certain forms of amyloid diseases and encompasses both monomeric and oligomeric proteins. Precursor amyloid proteins include, but are not limited to, Serum Amyloid A protein (ApoSSA), immunoglobulin light chain, immunoglobulin heavy chain, ApoA1, transthyretin, lysozyme, fibrinogen alpha chain, gelsolin, cystatin C, amyloid beta protein precursor (beta-APP), Beta₂ microglobulin, prion precursor protein (PrP), atrial natriuretic factor, keratin, islet amyloid polypeptide, a peptide hormone, and synuclein. The terms also encompass proteins having substantial similarity (as defined below) to amyloid proteins, such as, e.g., structural variants. The proteins may occur naturally or be synthetically constructed.

In the present context the terms “P”, “P peptide” or “peptide” are intended to mean both short peptides of from 2 to 10 amino acid residues, oligopeptides of from 11 to 100 amino acid residues, polypeptides of more than 100 amino acid residues, and full length proteins. Specifically, a P peptide consists of at least 3 amino acids from an amyloid protein, such as, e.g. beta amyloid The terms also encompass peptides having substantial similarity (as defined below) to amyloid proteins, such as, e.g., structural variants. The proteins may occur naturally or be synthetically constructed.

The term “substantial similarity” means that two peptide sequences, when optimally aligned, share at least 50 percent sequence identity, preferably at least 60 percent sequence identity, more preferably at least 70 percent sequence identity, more preferably at least 80 percent sequence identity, more preferably at least 90 percent sequence identity, more preferably at least 95 percent sequence identity or more (e.g., 99 percent sequence identity). Preferably, residue positions, which are not identical, differ by conservative amino acid substitutions. Conservative amino acid substitutions refer to the interchangeability of residues having similar side chains. For example, a group of amino acids having aliphatic side chains is glycine, alanine, valine, leucine, and isoleucine; a group of amino acids having aliphatic-hydroxyl side chains is serine and threonine; a group of amino acids having amide-containing side chains is asparagine and glutamine; a group of amino acids having aromatic side chains is phenylalanine, tyrosine, and tryptophan; a group of amino acids having basic side chains is lysine, arginine, and histidine; and a group of amino acids having sulfur-containing side chains is cysteine and methionine. Preferred conservative amino acids substitution groups are: valine-leucine-isoleucine, phenylalanine-tyrosine, lysine-arginine, alanine-valine, and asparagine-glutamine. Residue positions, which are not identical may also be composed of peptide analogs, including unnatural amino acids or derivatives of such. Analogs typically differ from naturally occurring peptides at one, two or a few positions, often by virtue of conservative substitutions. Some analogs also include unnatural amino acids or modifications of N or C terminal amino acids at one, two or a few positions. Examples of unnatural amino acids are D-amino acids, alpha, alpha-disubstituted amino acids, N-alkyl amino acids, lactic acid, 4-hydroxyproline, y-carboxyglutamate, epsilon-N,N,N-tri methyllysine, epsilon-N-acetyllysine, O-phosphoserine, N-acetylserine, N-formylmethionine, 3-methylhistidine, 5-hydroxylysine, .omega.-N-methylarginine, and isoaspartic acid.

The term “amyloid-induced cellular toxicity” describes a process of apoptotic or necrotic cell death induced in a population or subpopulation of cells, in vitro or in vivo, upon exposure to an amyloid protein or fragment thereof. The cell death is typically measured in vivo using histological stainings or in vitro using metabolic assays such as MTT or LDH

The terms “fibrils, plaques and/or amyloid deposits” means aggregated amyloid proteins, i.e. proteins that are not present as disaggregated or monomeric peptide units. Aggregated amyloid is a mixture of oligomers in which the monomeric units are held together by noncovalent bonds. In fibrils and plaques, all or part of the amyloid proteins are folded into beta sheet as can be visualised by staining for fibrillar amyloid using e.g. Congo red or Thioflavin S.

The term “immunological” or “Immune response” is the development of a beneficial humoral (antibody mediated) and/or a cellular (mediated by antigen-specific T cells or their secretion products) response directed against an amyloid peptide in a mammal. Such a response can be an active response induced by administration of immunogen or a passive response induced by administration of antibody or primed T-cells. A cellular immune response is elicited by the presentation of polypeptide epitopes in association with Class I or Class II MHC molecules to activate antigen-specific CD4.sup.+ T helper cells and/or CD8.sup.+ cytotoxic T cells. The relative contributions of humoral and cellular responses to the protective or therapeutic effect of an immunogen can be distinguished by separately isolating antibodies and T-cells from an immunized syngeneic animal and measuring protective or therapeutic effect in a second subject.

The term “amyloid related diseases” includes diseases associated with the accumulation of amyloid, which can either be restricted to one organ, i.e. “localized amyloidosis”, or spread to several organs, which is denoted “systemic amyloidosis”. Secondary amyloidosis may be associated with chronic infection (such as tuberculosis) or chronic inflammation (such as rheumatoid arthritis), including a familial form of secondary amyloidosis which is also seen in Familial Mediterranean Fever (FMF) and another type of systemic amyloidosis found in long-term hemodialysis patients. Localized forms of amyloidosis include, without limitation, diabetes type II and any related disorders thereof, neurodegenerative diseases such as scrapie, bovine spongiform encephalitis, Creutzfeldt-Jakob disease, Alzheimer's disease, Cerebral Amyloid Angiopathy, and prion protein related disorders. As mentioned above, the hallmark of amyloid diseases is the deposition in organs of amyloid plaques consisting mainly of fibrils, which, in turn, are composed of characteristic fibril proteins or peptides.

The term “antibody” describes proteins, which exhibit binding specificity to a specific antigen. The term is used in the broadest sense and covers monoclonal and polyclonal antibodies as well as antibody fragments.

The term “monoclonal antibody” is intended to mean an antibody obtainable from a population of substantially homogenous antibodies. Monoclonal antibodies are highly specific and are directed against a single determinant.

The term “polyclonal antibody” describes different antibodies, which are directed against different determinants.

The term “chimeric antibodies” is intended to mean an antibody in which a portion of the heavy and/or light chain is identical with or homologous to corresponding sequences in antibodies derived from a particular species or belonging to a particular antibody class, while the remainder of the chains is identical with or homologous to corresponding sequences in antibodies derived from another species or belonging to another antibody class. An example is an antibody wherein the antigen-binding site is derived from a mouse antibody, and the remainder of the antibody or fragment thereof is derived from a human antibody.

By the term “humanised antibody” is understood an antibody wherein only the complementarity determining regions, which are responsible for antigen binding and specificity are derived from a non-human species, and wherein the remainder of the antibody or fragment thereof is derived from human antibodies.

The term “epitope” refers to a site on an antigen to which B and/or T cells respond. B-cell epitopes can be formed both from contiguous amino acids or noncontiguous amino acids juxtaposed by tertiary folding of a protein. Epitopes formed from contiguous amino acids are typically retained on exposure to denaturing solvents whereas epitopes formed by tertiary folding are typically lost on treatment with denaturing solvents. An epitope typically includes at least 3, and more usually, at least 5 or 8-10 amino acids in a unique spatial conformation.

A suitable conjugate for targeting the pathological region(s) of amyloid proteins, and which may be used to develop both an active and a passive vaccine strategy, may be based on the Ligand Presenting Assembly (LPA) technology described in WO 00/18791. The technology is unique in making it possible to generate dimeric C-terminally-presented peptide fragments attached to a common backbone (see Formula I below and FIG. 1 for specific details). Conjugates of this type are highly immunogenic and capable of inducing specific immune responses. Using such a conjugate in an active vaccination strategy should generate an immune response of therapeutic level.

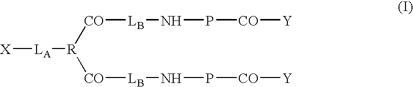

R represents the LPA linker, and may be selected from —N(CH₂—)₂, —NHCH< and —NHCH(CH₂—)₂. In a specific embodiment R is —N(CH₂—)₂.

X represents hydrogen or a peptidic group. Especially relevant peptidic groups according to the invention are such that may enhance the immune response in the mammal to which the conjugate is administered. Examples of suitable peptidic groups are T helper cell epitopes, which are peptides capable of binding to a MHC molecule and which stimulates T-cells in animal species.

In specific embodiments X may be a human T cell epitope including full-length tetanus toxoid, tetanus toxoid fragment FNNFTVSFWLRVPKVSASHLE and tetanus toxoid fragment YNDMFNNFTVSFWLRVPKVSASHLEQYGT, or a rodent T cell epitope including QYIKANSKFIGITEL. X may also be chosen among e.g. Keyhole Limpet Hemocyanin or BSA.

Even though X is defined above as being hydrogen or a peptidic group, in certain cases X may also be chosen among one of the following chemical groups: C₁₋₈alkyl, C₂₋₈alkenyl, C₂₋₈alkynyl, C₃₋₁₀cycloalkyl, C₅₋₁₀cycloalkenyl, C₃₋₇heterocycloalkyl, aryl, heteroaryl, C₁₋₈alkoxy, C₁₋₈alkylthio, C₁₋₈alkylcarboxy, C₁₋₈alkylcarbonyl, C₁₋₈alkylphosphonyl, aryloxy, arylthio, arylsulfonyl, arylsulfonate, arylcarboxy, arylcarbonyl, or aroyl. X may also be a dicyclic monocarboxylic acid such as, e.g. biotin.

If X is hydrogen or one of the chemical groups mentioned above, a suitable T helper cell epitope may be administered separately, but as part of the same vaccine regimen as the conjugate.

A linker L_{A} may optionally be present linking X and the R-group. L_{A} may be an amino acid or a peptide containing at least 2 amino acid residues. In a specific embodiment L_{A} is β-alanine.

The N-terminal end of P may be linked to R via a linker L_{B} that also represents an amino acid or a peptide containing at least 2 amino acid residues. The amino acids or peptides may be such which confer specific beneficial properties to P. In a specific embodiment L_{B} is a dipeptide consisting of two lysines (KK), for improving the solubility of P.

P is a peptide selected from full length or fragments of amyloid proteins or proteins with substantial similarity to an amyloid protein. As it appears from the formula above, the N-terminal end of P is linked to R or, if present, to L_{B} by an amide bond so that P is C-terminally presented. Accordingly, the —NH group between L_{B} and P intends to illustrate the orientation of P, and should be interpreted as the N-terminal of P and not as an additional —NH group. The same applies for the carbonyl (—CO) group between P and Y, which should be interpreted as the C-terminal group of P, and not as an additional —CO group.

P may be the same or different peptides, however, in most cases the two peptides are the same. As compared to peptides used in known vaccines the peptides according to the present invention need not to be aggregated to be operative or immunogenic.

P may be a fragment of an amyloid protein comprising at least one specific functional region. Such regions may be selected from the group comprising the C-terminal region, beta sheet region, cytotoxic region, GAG-binding site region, or macrophage adherence region. The conjugate may thereby be effective in inducing an immune response directed against a specific region or epitope formed by an amyloid protein or a fibril of an amyloid protein.

In the rare occasions where the two P peptides in the conjugate are different, they may be different fragments of the same amyloid protein and comprise different regions thereof. Another possibility is that the P peptides are different fragments of regions of different amyloid proteins.

In most occasions the C-terminal end group of P is a carboxylic acid group, i.e. Y is OH. However, in some occasions Y may be NH₂.

As mentioned above the present invention is based on the discovery that amyloid diseases can be treated by administering peptides that serve to stimulate an immune response against a component or components of the various disease-specific amyloid proteins. The sections below serve to exemplify major forms of amyloidosis and the related amyloid proteins or precursor proteins that may be used in a conjugate or method according to the invention and are not intended to limit the invention in any way.

The peptides or proteins forming the amyloid deposits are often produced from a larger precursor protein. More specifically, the pathogenesis of amyloid fibril deposits generally involves proteolytic cleavage of a precursor protein into fragments. These fragments generally aggregate into anti-parallel beta-pleated sheets; however, certain undegraded forms of precursor protein have been reported to aggregate and form fibrils in familial amyloid polyneuropathy (variant transthyretin fibrils) and dialysis-related amyloidosis.

Relevant amyloid precursor proteins are selected from the group comprising serum amyloid A protein (ApoSSA), immunoglobulin light chain, immunoglobulin heavy chain, ApoA1, transthyretin, lysozyme, fibrinogen alpha chain, gelsolin, cystatin C, amyloid beta protein precursor (beta.-APP), beta₂ microglobulin, prion precursor protein (PrP), atrial natriuretic factor, keratin, islet amyloid polypeptide and synuclein or any polypeptides with substantial similarity to any of the above. In the following is explained how these precursor proteins is involved in specific amyloid-related diseases.

AA (Reactive) Amyloidosis

Generally, AA amyloidosis is a manifestation of a number of diseases that provoke a sustained acute phase response. Such diseases include chronic inflammatory disorders, chronic local or systemic microbial infections, and malignant neoplasms.

AA fibrils are generally composed of 8000 dalton fragments (AA peptide or protein) formed by proteolytic cleavage of serum amyloid A protein (apoSSA), a circulating apolipoprotein which is present in HDL complexes and which is synthesized in hepatocytes in response to such cytokines as IL-1, IL-6 and TNF. Deposition can be widespread in the body, with a preference for parenchymal organs. The spleen is usually a deposition site, and the kidneys may also be affected. Deposition is also common in the heart and gastrointestinal tract.

AA amyloid diseases include, but are not limited to inflammatory diseases, such as rheumatoid arthritis, juvenile chronic arthritis, ankylosing spondylitis, psoriasis, psoriatic arthropathy, Reiter's syndrome, Adult Still's disease, Behcet's syndrome, and Crohn's disease. AA deposits are also produced as a result of chronic microbial infections, such as leprosy, tuberculosis, bronchiectasis, decubitus ulcers, chronic pyelonephritis, osteomyelitis, and Whipple's disease. Certain malignant neoplasms can also result in AA fibril ainyloid deposits. These include such conditions as Hodgkin's lymphoma, renal carcinoma, carcinomas of gut, lung and urogenital tract, basal cell carcinoma, and hairy cell leukemia.

AL Amyloidoses

AL amyloid deposition is generally associated with almost any dyscrasia of the B lymphocyte lineage, ranging from malignancy of plasma cells (multiple myeloma) to benign monoclonal gammopathy. At times, the presence of amyloid deposits may be a primary indicator of the underlying dyscrasia.

Fibrils of AL amyloid deposits are composed of monoclonal immunoglobulin light chains or fragments thereof. More specifically, the fragments are derived from the N-terminal region of the light chain (kappa or lambda) and contain all or part of the variable (V_{L}) domain thereof. Deposits generally occur in the mesenchymal tissues, causing peripheral and autonomic neuropathy, carpal tunnel syndrome, macroglossia, restrictive cardiomyopathy, arthropathy of large joints, immune dyscrasias, myelomas, as well as occult dyscrasias. However, it should be noted that almost any tissue, particularly visceral organs such as the heart, may be involved.

Hereditary Systemic Amyloidoses

There are many forms of hereditary systemic amyloidoses. Although they are relatively rare conditions, adult onset of symptoms and their inheritance patterns (usually autosomal dominant) lead to persistence of such disorders in the general population Generally, the syndromes are attributable to point mutations in the precursor protein leading to production of variant amyloidogenic peptides or proteins. Without limiting the scope of the invention, some prominent example of this group is described in the following.

More than 40 separate point mutations in the transthyretin gene have been described, all of which give rise to clinically similar forms of familial amyloid polyneuropathy. Transthyretin (TTR) is a 14 kilodalton protein that is also sometimes referred to as prealbumin. It is produced by the liver and choroid plexus, and it functions in transporting thyroid hormones and vitamin A. At least 50 variant forms of the protein, each characterized by a single amino acid change, are responsible for various forms of familial amyloid polyneuropathy. For example, substitution of proline for leucine at position 55 results in a particularly progressive form of neuropathy; substitution of methionine for leucine at position 111 resulted in a severe cardiopathy in Danish patients. Amyloid deposits isolated from heart tissue of patients with systemic amyloidosis have revealed that the deposits are composed of a heterogeneous mixture of TTR and fragments thereof, collectively referred to as ATTR, the full length sequences of which have been characterized. ATTR fibril components can be extracted from such plaques and their structure and sequence determined according to the methods known in the art.

Persons having point mutations in the molecule apolipoprotein AI (e.g., Gly.fwdarw.Arg26; Trp 4.fwdarw.Arg50; Leu.fwdarw.4 Arg60) exhibit a form of amyloidosis (“stertag type”) characterized by deposits of the protein apolipoprotein AI or fragments thereof (AApoAI). These patients have low levels of high density lipoprotein (HDL) and present with a peripheral neuropathy or renal failure.

A mutation in the alpha chain of the enzyme lysozyme (e.g., lle.fwdarw.Thr56 or Asp.fwdarw.His57) is the basis of another form of stertag-type non-neuropathic hereditary amyloid reported in English families. Here, fibrils of the mutant lysozyine protein (Alys) are deposited, and patients generally exhibit impaired renal function. This protein, unlike most of the fibril-forming proteins described herein, is usually present in whole (unfragmented) form.

Amyloid beta (Aβ) is a 39-43 amino acid peptide derived by proteolysis from a large protein known as Beta Amyloid Precursor protein (βAPP). Mutations in βAPP result in familial forms of Alzheimer's disease, characterized by cerebral deposition of plaques composed of βP fibrils and other components, which are described in further detail below. Known mutations in APP associated with Alzheimer's disease occur proximate to the cleavage sites of β- or gamma-secretase, or within Aβ. For example, position 717 is proximate to the site of gamma-secretase cleavage of APP in its processing to Aβ, and positions 670/671 are proximate to the site of β-secretase cleavage. Mutations at any of these residues may result in Alzheimer's disease, presumably by causing an increase the amount of the highly amyloidogenic 42/43 amino acid form of Aβ generated from APP. The structure and sequence of Aβ peptides of various lengths are well known in the art. Such peptides can be made according to methods known in the art. In addition, various forms of the peptides are commercially available.

Synuclein is a synapse-associated protein that resembles an alipoprotein and is abundant in neuronal cytosol and presynaptic terminals. A peptide fragment derived from alpha-synuclein, termed NAC, is also a component of amyloid plaques of Alzheimer's disease. This component also serves as a target for immunologically-based treatments of the present invention, as detailed below. Fibrils of full length synuclein and NAC are also intrinsic components of Lewy bodies that are associated with Parkinson's disease.

Gelsolin is a calcium binding protein that binds to fragments and actin filaments. Mutations at position 187 (e.g., Asp.fwdarw.Asn; Asp.fwdarw.Tyr) of the protein result in a form of hereditary systemic amyloidosis, usually found in patients from Finland, as well as persons of Dutch or Japanese origin. In afflicted individuals, fibrils formed from gelsolin fragments (Agel), usually consist of amino acids 173-243 (68 kDa carboxyterminal fragment) and are deposited in blood vessels and basement membranes, resulting in corneal dystrophy and cranial neuropathy which progresses to peripheral neuropathy, dystrophic skin changes and deposition in other organs.

Other mutated proteins, such as mutant alpha chain of fibrinogen (AfibA) and mutant cystatin C (Acys) also form fibrils and produce characteristic hereditary disorders. AfibA fibrils form deposits characteristic of a nonneuropathic hereditary amyloid with renal disease; Acys deposits are characteristic of a hereditary cerebral amyloid angiopathy reported in Iceland. In at least some cases, patients with cerebral amyloid angiopathy (CAA) have been shown to have amyloid fibrils containing a non-mutant form of cystatin C in conjunction with beta protein.

Certain forms of prion diseases are now considered to be heritable, accounting for up to 15% of cases, which were previously thought to be predominantly infectious in nature. In such prion disorders, patients develop plaques composed of abnormal isoforms of the normal prion protein (PrP_{Sc}). A predominant mutant isoform, PrP_{Sc}, also referred to as AScr, differs from the normal cellular protein in its resistance to protease degradation, insolubility after detergent extraction, deposition in secondary lysosomes, post-translational synthesis, and high β-pleated sheet content. Genetic linkage has been established for at least five mutations resulting in Creutzfeldt-Jacob disease (CJD), Gerstmann-Strussler-Scheinker syndrome (GSS), and fatal familial insomnia (FFI). Methods for extracting fibril peptides from scrapie fibrils, determining sequences and making such peptides are known in the art.

Senile Systemic Amyloidosis

Amyloid deposition, either systemic or focal, increases with age. For example, fibrils of wild type transthyretin (TTR) are commonly found in the heart tissue of elderly individuals. These may be asymptomatic, clinically silent, or may result in heart failure. Asymptomatic fibrillar focal deposits may also occur in the brain (Aβ), corpora amylacea of the prostate (Aβ₂ microglobulin), joints and seminal vesicles.

Cerebral Amyloidosis

Local deposition of amyloid is common in the brain, particularly in elderly individuals. The most frequent type of amyloid in the brain is composed primarily of Aβ peptide fibrils, resulting in dementia or sporadic (non-hereditary) Alzheimer's disease. In fact, the incidence of sporadic Alzheimer's disease greatly exceeds forms shown to be hereditary. Fibril peptides forming these plaques are very similar to those described above, with reference to hereditary forms of Alzheimer's disease (AD).

Dialysis-Related Amyloidosis

Plaques composed of β₂ microglobulin (Aβ₂M) fibrils commonly develop in patients receiving long term hemodialysis or peritoneal dialysis. β₂ microglobulin is a 11.8 kilodalton polypeptide and is the light chain of Class I MHC antigens, which are present on all nucleated cells. Under normal circumstances, it is continuously shed from cell membranes and is normally filtered by the kidney. Failure of clearance, such as in the case of impaired renal function, leads to deposition in the kidney and other sites (primarily in collagen-rich tissues of the joints). Unlike other fibril proteins, A β₂M molecules are generally present in unfragmented form in the fibrils.

Hormone-Derived Amyloidoses

Endocrine organs may harbor amyloid deposits, particularly in aged individuals. Hormone-secreting tumors may also contain hormone-derived amyloid plaques, the fibrils of which are made up of polypeptide hormones such as calcitonin (medullary carcinoma of the thyroid), islet amyloid polypeptide (amylin; occurring in most patients with Type II diabetes), and atrial natriuretic peptide (isolated atrial amyloidosis). Sequences and structures of these proteins are well known in the art.

Miscellaneous Amyloidoses

There are a variety of other forms of amyloid disease that are normally manifest as localized deposits of amyloid. In general, these diseases are probably the result of the localized production and/or lack of catabolism of specific fibril precursors or a predisposition of a particular tissue (such as the joint) for fibril deposition. Examples of such idiopathic deposition include nodular AL amyloid, cutaneous amyloid, endocrine amyloid, and tumor-related amyloid.

Accordingly, conjugates for use in the treatment of e.g. hereditary forms of amyloidosis as discussed above, may contain and thereby being capable of generating antibodies towards the following: gelsolin fragments for treatment of hereditary systemic amyloidosis, mutant lysozyme protein (Alys) for treatment of a hereditary neuropathy, mutant alpha chain of fibrinogen (AfibA) for a non-neuropathic form of amyloidosis manifest as renal disease and mutant cystatin C (Acys) for treatment of a form of hereditary cerebral angiopathy reported in Iceland. In addition, certain hereditary forms of prion disease (e.g., Creutzfeldt-Jacob disease (CJD), Gerstmann-Strussler-Scheinker syndrome (GSS), and fatal familial insomnia (FFI)) are characterized by a mutant isoform of prion protein, PrP.sup.Sc, and this protein may be used in conjugates for treatment and prevention of deposition of PrP plaques in accordance with the present invention.

By way of further example, but not limitation, there are a number of additional, non-hereditary forms amyloid disease that are candidates for use in conjugates according to the present invention. Beta₂ microglobulin fibrillar plaques commonly develop in patients receiving long term hemodialysis or peritoneal dialysis. Such patients may be treated with conjugates directed to beta₂ microglobulin or, more preferably, immunogenic epitopes thereof, in accordance with the present invention.

Hormone-secreting tumors may also contain hormone-derived amyloid plaques, the composition of which is generally characteristic of the particular endocrine organ affected. Thus such fibrils may be made up of polypeptide hormones such as calcitonin (medullary carcinoma of the thyroid), islet amyloid polypeptide (occurring in most patients with Type II diabetes), and atrial natriuretic peptide (isolated atrial amyloidosis). Conjugates directed at amyloid deposits which form in the aortic intima in atherosclerosis are also contemplated by the present invention.

In the following the invention is exemplified by the amyloid protein amyloid beta.

Amyloid beta is the main constituent of the amyloid plaques and deposits formed in e.g. Alzheimer's disease (AD). Alzheimer's disease is characterized by the deposition of neuritic plaques and neurofibrillary tangles.

There exist no direct correlation between neurological deficit and amyloid deposit burden. The lack of correlation is both evident in human AD patients, and in transgenic mice models of AD. Several lines of evidence also indicate that immobilized amyloid plaques are relatively harmless structures, whereas soluble amyloid beta oligomers are responsible for most of the neurotoxic and inflammatory damage associated with amyloid beta. Soluble amyloid beta oligomers have also been implicated in the physical degeneration of synapses.

Accordingly, treatment of AD and other amyloid beta-related diseases should therefore most likely aim at removing soluble amyloid beta oligomers, whereas immobilized and therefore harmless amyloid beta in amyloid plaques could be left intact.

Oligomerisation of amyloid beta begins with the formation of monomeric beta-sheet structures, and this process is strongly enhanced by the presence of the C-terminus of amyloid beta. The primary goals for a prophylactic treatment of Alzheimer's disease may therefore be to develop an active and/or passive vaccination strategy specifically targeting the pathological C-terminus of amyloid beta and without the undesirable inflammatory properties seen in previous vaccination regimens.

Accordingly, the P peptides in a conjugate according to the invention may be selected from full length or a fragment of the human forms amyloid beta which are referred to as amyloid beta (1-43), amyloid beta (1-42), amyloid beta (1-41), amyloid beta (1-40), amyloid beta (1-39) and amyloid beta (1-38). Amyloid beta (1-42) has the following sequence:

H2N-Asp-Ala-Glu-Phe-Arg-His-Asp-Ser-Gly-Tyr-Glu-Val-His-His-Gln-Lys-Leu-Val-Phe-Phe-Ala-Glu-Asp-Val-Gly-Ser-Asn-Lys-Gly-Ala-Ile-Ile-Gly-Leu-Met-Val-Gly-Gly-Val-Val-Ile-Ala-OH

Amyloid beta (1-41), amyloid beta (1-40) and amyloid beta (1-39) differ from amyloid beta (1-42), by the omission of Ala, Ala-Ile and Ala-Ile-Val respectively from the C-terminal end. Amyloid beta (1-43) differs from amyloid beta (1-42) by the presence of a threonine residue at the C-terminus.

In a preferred embodiment P may contain the C-terminus of amyloid beta, such as, e.g. 10, 9, 8, 7, 6, 5, 4 or 3 amino acids from the C-terminus of amyloid beta.

Specifically, amyloid beta (1-42) is known to display a strong tendency to rapidly form amyloid fibrils, whereas shorter forms of amyloid beta peptides have a significantly weaker tendency to form fibrils. It is therefore likely that amyloid beta (1-42) is catalyzing the formation of amyloid fibrils in Alzheimer's disease and by specifically eliminating soluble amyloid beta (1-42) immunologically, it may be possible to prevent or alleviate the pathology of Alzheimer's disease.

The present inventors have identified very specific fragments of the C-terminal part of amyloid beta (1-42), that when administered to a mammal generates antibodies, which specifically target the synaptotoxic, soluble form of amyloid beta (1-42). There are several advantages by using a conjugate comprising small C-terminal amyloid beta peptides as immunogen. Firstly, a more controllable immune response is generated than what is known from other vaccines, as the B-cell epitopes in the present conjugate is dependent on the free C-terminus of amyloid beta (1-42). This eliminates any risk of generating antibodies with cross reactivity to full length APP or immobilized amyloid beta peptides, which significantly reduces the risk of igniting an unspecific inflammatory response caused by releasing the large pool of amyloid beta that is bound in amyloid plaques. Secondly, the immunogens are unlikely to contain CD8 cell epitopes due to their small size. Moreover, the present inventors can avoid the Aβ₂₅₋₃₅ segment in their vaccine approach, thereby eliminating any unwanted neurotoxic or inflammatory effects, as the neurotoxic properties of amyloid beta is primarily located within the decapeptide Aβ₂₅₋₃₅, which also can bind to C1q and trigger the complement cascade.

Accordingly, in a specific embodiment the present invention relates to a conjugate, wherein P is fragment 35-42 of amyloid beta (1-42). The invention also relates to a conjugate, wherein P is fragment 36-42 of amyloid beta (1-42). Furthermore, the invention relates to a conjugate, wherein P is fragment 37-42 of amyloid beta (1-42). In yet another aspect, P is fragment 38-42 of amyloid beta (1-42). Moreover, the invention relates to a conjugate, wherein P is fragment 39-42 of amyloid beta (1-42). The invention also encompasses conjugates, wherein P is fragment 40-42 of amyloid beta (1-42).

Small peptides corresponding to the C-terminus of Aβ₁₋₄₂ are relatively poor immunogens, unlikely to induce a substantial immune response in isolation. To compensate for this, the inventors have used the proprietary LPA technology, described in WO 00/18791. Conjugates of this type are shown herein to be highly immunogenic and capable of inducing specific immune responses. Using such a conjugate in an active vaccination strategy should generate an immune response of therapeutic level, specifically targeting the pathogenic C-terminal part of Aβ₁₋₄₂.

In a preferred embodiment, R in Formula (I) is —N(CH₂—)₂, P is fragment 36-42 of amyloid beta (1-42) and X is a suitable T helper cell epitope, such as full length tetanus toxoid. Furthermore, L_{B}, if present, is two lysine residues (KK), and L_{A}, if present, is beta alanine, and Y is OH.

In another preferred embodiment, R is —N(CH₂—)₂, P is fragment 37-42 of amyloid beta (1-42) and X is a suitable T helper cell epitope, such as full length tetanus toxoid. Furthermore, L_{B}, if present, is two lysine residues (KK), and L_{A}, if present, is beta alanine, and Y is OH.

In yet another preferred embodiment, R is —N(CH₂—)₂, P is fragment 38-42 of amyloid beta (1-42) and X is a suitable T helper cell epitope, such as full length tetanus toxoid. Furthermore, L_{B}, if present, is two lysine residues (KK), and L_{A}, if present, is beta alanine, and Y is OH.

The P peptides according to the invention can be synthesized by solid phase peptide synthesis or recombinant expression, or can be obtained from natural sources. Automatic peptide synthesizers are commercially available from numerous suppliers, such as Applied Biosystems, Foster City, Calif. Recombinant expression can be in bacteria, such as E. coli, yeast, insect cells or mammalian cells. Procedures for recombinant expression are described by Sambrook et al., Molecular Cloning: A Laboratory Manual (C.S.H.P. Press, N.Y. 2d ed., 1989).

The conjugates may be synthesized by a method as described in WO 00/18791, and illustrated in FIG. 1 herein.

The invention also relates to an active immunization method for the treatment, amelioration and/or prophylaxis of an amyloid-related disease in a mammal, such as, e.g., Alzheimer's disease, Down's syndrome, vascular dementia or cognitive impairment, the method comprising administering to the mammal an antigenic amount of a conjugate as defined above, wherein the conjugate elicits the production of antibodies having specificity towards the conjugate itself and induces an immune response in the mammal, thereby preventing or reducing amyloid-induced cellular toxicity and/or the formation of fibrils, plaques and/or amyloid deposits.

In a specific embodiment, the antibodies produced are having specificity towards one or more C-terminally presented P peptides of a conjugate above, and accordingly, are capable of interacting with pathological regions within an amyloid protein. In a preferred embodiment, the conjugate is one of the specific conjugates described above comprising fragments of amyloid beta (1-42). The invention also relates to a method, which further comprises the administration of an adjuvant e.g. as defined below together with the conjugate.

In a method according to the invention the conjugate should be administered in a dosage effective enough to produce a suitable immune response against the relevant amyloid peptide characteristic of the amyloid disorder from which the subject suffers. In one embodiment the immunological response is characterized by a serum titer of at least 1:1000 with respect to the amyloid protein against which the immunogenic peptide is directed. In yet a further embodiment, the serum titer is at least 1:5000 with respect to the amyloid protein. According to a related embodiment, the immune response is characterized by a serum amount of immunoreactivity corresponding to greater than about four times higher than a serum level of immunoreactivity measured in a pre-treatment control serum sample. This latter characterization is particularly appropriate when serum immunoreactivity is measured by ELISA techniques, but can apply to any relative or absolute measurement of serum immunoreactivity. According to a preferred embodiment, the immunoreactivity is measured at a serum dilution of about 1:100.

A person skilled in the art will be able to determine a suitable dosage for a mammal in need thereof, based on the conjugates used, the conditions to be treated, and the weight, age and gender of the mammal in question. It is contemplated that suitable dosages will be in the range of ng to mg per day.

The invention also relates to a vaccine comprising a conjugate as described above together with an adjuvant. The vaccines according to the present invention cause the generation of effective anti-amyloidogenic antibodies in the vaccinated host.

The adjuvant is administered in order to augment the immune responses or to increase the antigenicity of antigenic conjugaet. Adjuvants exert their immunomodulatory properties through several mechanisms such as lymphoid cells recruitment and cytokine induction. Cytokine adjuvants include, without limitation, granulocyte-macrophage colony-stimulating factor, interleukin-12, GM-CSF, synthetic muramyl dipeptide analog or monophosphoryl lipid A. Further examples of adjuvants is selected from the group comprising complete Freunds adjuvant, incomplete Freunds adjuvant, QS21, aluminium hydroxide gel, MF59, calcium phosphate, liposyn, saponin, squalene, L121, emulsigen monophosphyryl lipid A (MPL), polysorbate 80, cholera toxin (CT), LTK and LTK63.

In a specific embodiment, the adjuvants are such, which are approved for administration to humans, such as aluminium hydroxide gel, calcium phosphate and MF59.

In another embodiment the adjuvant are of a type that stimulates a Th2 type of immune response, such as, e.g, aluminium hydroxide gel and CT. By inducing a Th2 type response, anti-inflammatory cytokine production such as IL-4, I1-10 and TGF-beta, as well as the production of IgG₁ and IgG_{2b} antibody classes, are favored. A Th2 type response may be preferred in the present invention, as major inflammatory responses in the brain of the patients with AD will be avoided, as IgG₁ is only modestly complement activating in humans. For a safer Alzheimer vaccine it is essential that the antibodies are not complement activating since such an immune response can mediate lyses of cells coated by the antibodies.

The vaccine may also comprise a suitable carrier including, without limitation, any non-immunogenic substance suitable for oral, parenteral, intravascular (IV), intranasal (IN), intraarterial (IA), intramuscular (IM), transdermal and subcutaneous (SC) administration routes, such as, e.g. phosphate buffer saline (PBS).

The carriers may also be vehicles, which carry antigens to antigen-presenting cells. Examples of vehicles are liposomes, immune-stimulating complexes, microfluidized squalene-in-water emulsions, microspheres which may be composed of poly(lactic/glycolic) acid (PLGA).

The description above has been focused on the development of an active vaccination approach, but a passive immuno-therapy approach may be developed along the same lines. The same conjugates as described above may be used to generate highly specific e.g. monoclonal antibodies. Using genetic manipulation, such antibodies could subsequently be humanized for further use in a passive vaccination strategy in humans. Using passive immunization as an approach would obviously have added safety advantages, as it would further eliminate the risk of inducing any unwanted inflammatory responses.

Accordingly, the invention also encompasses a passive immunization strategy for the treatment, amelioration and/or prophylaxis of an amyloid-related disease, such as, e.g., Alzheimer's disease, Down's syndrome, vascular dementia or cognitive impairment, in a mammal, wherein an antibody which interacts with amyloid proteins associated with the disease of which the mammal suffer is administered in an effective dose to the mammal thereby preventing or reducing amyloid-induced cellular toxicity and/or the formation of fibrils, plaques and/or amyloid deposits.

The antibody may be raised against a conjugate comprising one or more carboxyl-terminally exposed peptide as described above. In a specific embodiment the antibody binds to the carboxyl terminus of the amyloid beta (1-42), such as residues 35-42, 36-42, 37-42 or 38-42.

The antibody may also be a monoclonal antibody. In a preferred embodiment, a monoclonal antibody is produced by immunizing a mammalian subject with an amyloid peptide, e.g. amyloid beta(1-42) or fragments thereof linked to a LPA backbone and subsequently establish antibody producing cell lines by e.g. generating hybridoma cells by somatic cell hybridization using standard immunological techniques.

The antibody may be humanized or chimeric. Chimeric and humanized antibodies have the same or similar binding specificity and affinity as a mouse or other nonhuman antibody. Mammalian nonhuman antibodies provide the starting material for construction of a chimeric antibody. Chimeric antibodies are antibodies whose light and heavy chain genes have been constructed, typically by genetic engineering, from immunoglobulin gene segments belonging to different species. For example, the variable (V) segments of the genes from a mouse monoclonal antibody may be joined to human constant (C) segments, such as IgG1 and IgG4. Human isotype IgG1 is preferred. A typical chimeric antibody is thus a hybrid protein consisting of the V or antigen-binding domain from a mouse antibody and the C or effector domain from a human antibody.

Humanized antibodies can be generated using two different routes, either by molecular engineering of a mammalian nonhuman antibody or by raising an immune resonse in humanized mice, i.e transgenic mice with a human-like immune system. The molecular engineered antibodies use mammalian nonhuman antibodies as the starting material. They have variable region framework residues substantially from a human antibody (termed an acceptor antibody) and complementarity determining regions substantially from a mouse-antibody, (referred to as the donor immunoglobulin). The constant region(s), if present, are also substantially or entirely from a human immunoglobulin. The human variable domains are usually chosen from human antibodies whose framework sequences exhibit a high degree of sequence identity with the murine variable region domains from which the CDRs were derived. The heavy and light chain variable region framework residues can be derived from the same or different human antibody sequences. The human antibody sequences can be the sequences of naturally occurring human antibodies or can be consensus sequences of several human antibodies. Certain amino acids from the human variable region framework residues are selected for substitution based on their possible influence on CDR conformation and/or binding to antigen. Investigation of such possible influences is by modeling, examination of the characteristics of the amino acids at particular locations, or empirical observation of the effects of substitution or mutagenesis of particular amino acids.

Other candidates for substitution are acceptor human framework amino acids that are unusual for a human immunoglobulin at that position. These amino acids can be substituted with amino acids from the equivalent position of the mouse donor antibody or from the equivalent positions of more typical human immumoglobulins. Other candidates for substitution are acceptor human framework amino acids that are unusual for a human immunoglobulin at that position. The variable region frameworks of humanized immunoglobulins usually show at least 85% sequence identity to a human variable region framework sequence or consensus of such sequences.

Vaccines and passive immunization strategies according to the present invention, wherein P is a fragment of amyloid beta, are able to prevent the development of brain amyloidosis through two possible scenerios: 1) the effect of the anti-amyloid beta antibodies at the site of amyloid deposition, and 2) the systemic effect of the high circulatory anti-amyloid level on the plasmatic amyloid concentrations.

Specifically, elevated plasma anti-amyloid beta antibody levels may act systemically by decreasing normal amyloid beta plasma levels, thereby creating a systemic imbalance in the normal amyloid beta levels. Such an imbalance could lead to the activation of mechanisms responsible for the clearing in amyloid beta levels from the brain, in order to re-establish the normal balance between brain and plasma amyloid beta levels.

Accordingly, this possibility could be exploited by determining the effect of active or passive immunization on plasma and brain levels of e.g., amyloid beta (1-40) and amyloid beta (1-42) at different timepoints following such immunization. Amyloid beta-immunization can also exert a systemic protective effect versus the development of brain amyloidosis. The ratio of Amyloid beta levels in plasma and brain should remain constant in immunized transgenic animals, while it should decrease in the control animals. Additionally, B-cell or bone marrow cell transfer from immunized to naive transgenic animals should have the same effect as passive immunization using anti-amyloid beta antibodies.

The vaccines and antibodies according to the present invention will, for the most part, be administered parenterally, such as intravascularly (IV), intraarterially (IA), intramuscularly (IM), subcutaneously (SC), or the like. In some instances, administration may be oral, nasal, rectal, transdermal or aerosol, where the nature of the vaccine allows for transfer to the vascular system. Usually a single injection will be employed although more than one injection may be used, if desired. The vaccine may be administered by any convenient means, including syringe, trocar, catheter, or the like. Preferably, the administration will be intravascularly, where the site of introduction is not critical to this invention, preferably at a site where there is rapid blood flow, e.g., intravenously, peripheral or central vein. Other routes may find use where the administration is coupled with slow release techniques or a protective matrix. Also, mucosal immunization via nasal administration is a suitable method, since it is known that such a route of administration would favor a Th2 type response.

In alternative embodiments of the present invention, the linker R connecting the one or more P peptides and the X group may be different than defined above. In such cases, P represents only a very limited number of peptides as defined in the following items:

1. A conjugate having the following structure
X—R-(L_{B}-P)ₙ
wherein

X is hydrogen or a peptidic group,

R is a linker having at least two attachments points and being capable of forming a covalent bond to the N-terminal end of P, or via L_{B} if relevant,

n is an integer higher than or similar to 1, and

P is a peptide containing the C-terminal of amyloid beta and wherein the N-terminal end of P is linked to L or, if present, to X by covalent bond so that P is C-terminally presented and when n is 2 or more then P is the same or different,

and pharmaceutically acceptable salts thereof.

2. A conjugate according to item 1, wherein P, or at least one of P if n is two or more, is a fragment of 10 amino acids from the C-terminus of amyloid beta.

3. A conjugate according to item 1, wherein P, or at least one of P if n is two or more is a fragment of 9 amino acids from the C-terminus of amyloid beta.

4. A conjugate according to item 1, wherein P, or at least one of P if n is two or more is a fragment of 8 amino acids from the C-terminus of amyloid beta.

5. A conjugate according to item 1, wherein P, or at least one of P if n is two or more is a fragment of 7 amino acids from the C-terminus of amyloid beta.

6. A conjugate according to item 1, wherein P, or at least one of P if n is two or more is a fragment of 6 amino acids from the C-terminus of amyloid beta.

7. A conjugate according to item 1, wherein P, or at least one of P if n is two or more is a fragment of 5 amino acids from the C-terminus of amyloid beta.

8. A conjugate according to item 1, wherein P, or at least one of P if n is two or more is a fragment of 4 amino acids from the C-terminus of amyloid beta.

9. A conjugate according to item 1, wherein P, or at least one of P if n is two or more is a fragment of 3 amino acids from the C-terminus of amyloid beta.

10. A conjugate according to item 1, wherein P, or at least one of P if n is two or more, is fragment 35-42 of amyloid beta (1-42).

11. A conjugate according to item 1, wherein P, or at least one of P if n is two or more, is fragment 36-42 of amyloid beta (1-42).

12. A conjugate according to item 1, wherein P, or at least one of P if n is two or more, is fragment 37-42 of amyloid beta (1-42).

13. A conjugate according to item 1, wherein P, or at least one of P if n is two or more, comprises fragment 38-42 of amyloid beta (1-42).

14. A conjugate according to item 1, wherein P, or at least one of P if n is two or more, comprises fragment 39-42 of amyloid beta (1-42).

15. A conjugate according to item 1, wherein P, or at least one of P if n is two or more, comprises fragment 40-42 of amyloid beta (1-42).

16. A conjugate according to any of items 1-15, wherein n is 2 and P is the same peptide.

17. A conjugate according to any of items 1-16, wherein the linker Y is selected from any suitable type as described in Aslam, Protein Coupling Techniques for the Biomedical Sciences, (1998).

18. A conjugate according to any of items 1-17, wherein R is a T cell epitope.

19. A conjugate according to item 18, wherein R is a human T cell epitope including full-length tetanus toxoid, tetanus toxoid fragment FNNFTVSFWLRVPKVSASHLE and tetanus toxoid fragment YNDMFNNFTVSFWLRVPKVSASHLEQYGT, or a rodent T cell epitope including QYIKANSKFIGITEL.

20. A conjugate according to any of items 1-17, wherein R is Keyhole Limpet Hemocyanin or BSA.

21. A method for producing an antibody in a mammal, the method comprising administering to the mammal an antigenic amount of a conjugate as defined in any of the items 1-20, wherein the conjugate elicits the production of antibodies having specificity towards the conjugate itself.

22. A method according to item 21, wherein antibodies produced are being specific towards one or more C-terminally presented P peptides of a conjugate as defined in claims 1-20.

23. A method according to item 21 or 22, which further comprises the step of generating hybridoma cells by somatic cell hybridization for the production of monoclonal or polyclonal antibodies.

24. A method according to any of items 21-23, wherein the mammal is a mouse or humanized mouse.

25. An antibody having specificity towards a conjugate as defined in items 1-20.

26. An antibody having specificity towards one or more C-terminally presented P peptides in a conjugate as defined in any of items 1-20.

27. An antibody according to item 25 or 26, which is monoclonal.

28. An antibody according to any of items 25-27, which is humanized or chimeric.

29. An antibody according to any of items 25-28, which is produced by a method as defined in items 21-24.

30. A method for the treatment and/or prophylaxis of an amyloid-related disease in a mammal, the method comprising administering to the mammal an antibody as defined in items 25-29, thereby preventing or reducing amyloid-induced cellular toxicity and/or the formation of fibrils, plaques and/or amyloid deposits.

### FIGURE LEGENDS

FIG. 1 illustrates the generation of dimeric C-terminally-presented peptide fragments attached to a common backbone. 1) The Aβ peptide sequence was assembled on the synthesis resin. The chains further comprise two N-terminal lysines for hydrofilicity enhancement. Only two of many chains are shown. 2) ½ equivalent of Fmoc-imino diacidic acid were coupled to the N-terminus of he peptide chains with TBTU, HOBt and DIEA as coupling reagents. 3) Cyclisation was carried out using TBTU. 4) De-protection and cleavage from the resin results in the biologically enhanced dimer with C-terminal presentation. 5) N-terminal de-protection of (3) and subsequently coupling with Fmoc-βAla-OH to the imino-group (NH-group) a spacer provides a peptide resin for continued synthesis of a T cell epitope at the NH-group. De-protection and cleavage from the resin results in the dimer.

FIG. 2 shows a histogram of the proliferative response. Mice were immunized with Alhydrogel, p30ex in Alhydrogel or tetanus toxoid in Alhydrogel, spleen cells were obtained and stimulated with p30ex (blue) or tetanus toxoid (red).

FIG. 3 shows a histogram of the proliferative response. Peripheral blood mononuclear cells (PBMC) from an individual was immunized with tetanus vaccine, and PBMC were obtained and stimulated with tetanus toxoid, p30ex or no antigen.

FIG. 4 shows ELISA with Aβ₃₃₋₄₂ as antigen using serum dilutions of 1:50. Mice were immunized with P30ex:LPA-KK:(³³GLMVGGVVIA⁴²)₂ (IgG aa 33-42), P30ex:LPA-KK:(³⁵MVGGVVIA⁴²)₂ (IgG aa 35-42), P30ex:LPA-KK:(³⁶VGGVVIA⁴²)₂ (IgG aa 36-42), P30ex:LPA-KK:(³⁷GGVVIA⁴²)₂ (IgG aa 37-42), P30ex:LPA-KK:(³⁸GVVIA⁴²)₂ (IgG aa 38-42) and Alhydrogel (IgG Alhydrogel).

FIG. 5 shows ELISA with Aβ₃₃₋₄₂ as antigen. Mice were immunized with KLH:LPA-KK:(³⁷GGVVIA⁴²)₂ (IgG aa 37-42 KLH).

FIG. 6 is a IgG₁/IgG₂ₐ subclass ELISA of the P30ex:LPA-KK:(³³GLMVGGVVIA⁴²)2 group.

FIG. 7 is a IgG₁/IgG₂ₐ subclass ELISA of the P30ex:LPA-KK: (³⁵MVGGVVIA⁴²)₂ group.

FIG. 8 is a IgG_{1/IgG}₂ₐ subclass ELISA of the KLH:LPA-KK:(³⁷MGGVVIA⁴²)₂ group.

FIG. 9 shows ELISA for determination of the selectivity of the immune response to P30ex:LPA-KK:(³³GLMVGGVVIA⁴²)₂ antigen using Aβ₃₃₋₄₂ and Aβ₃₃₋₄₄ as coating antibodies.

FIG. 10 shows ELISA for determination of the selectivity of the immune response to the P30ex:LPA-KK:(³⁵MVGGVVIA⁴²)₂ antigen using Aβ₃₃₋₄₂ and Aβ₃₃₋₄₄ as coating antibodies.

FIG. 11 shows ELISA for the determination of the selectivity of the immune response to the KLH:LPA-KK:(³⁷GGVIA⁴²)₂ antigen using Aβ₃₃₋₄₂ and Aβ₃₃₋₄₄ as coating antibodies.

FIG. 12 illustrates spleen cell proliferation. Spleen cells from mice immunised with KLH:LPA-KK:(³⁷GGVVIA⁴²)₂ were re-stimulated in vitro with KLH (blue) and Aβ₁₋₄₂ (Red). Background proliferation is shown in yellow.

### EXAMPLES

### Example 1

### Generation of Antigen Constructs

Using the LPA technology described below five antigen constructs were designed. The constructs were all composed of a T cell epitope, the LPA backbone, two lysine (K) and two C-terminal peptide fragment of Aβ₁₋₄₂ (see Table I). The Aβ sequences in the antigen constructs were decreasing in length from 10 to 5 amino acids, Aβ_{33/35/36/37/38-42}.

TABLE I

Overview of generated antigen constructs

Antigen

| | | |
|---|---|---|
| P30ex:LPA-KK: | (³³GLMVGGVVIA⁴²)₂ | (NSA) |
| P30ex:LPA-KK: | (³⁵MVGGVVIA⁴²)₂ | (NSB) |
| P30ex:LPA-KK: | (³⁶VGGVVIA⁴²)₂ | (NSC) |
| P30ex:LPA-KK: | (³⁷GGVVIA⁴²)₂ | (NSD) |
| P30ex:LPA-KK: | (³⁸GVVIA⁴²)₂ | (NSE) |

Peptide Synthesis

Peptides were synthesized on a fully automatic ABI 433 peptide synthesis instrument (Applied Biosystems) using Fmoc-amino acids (Fluka) with TBTU (N,N,N′,N′-tetramethyl-O-benzotriazol-1-yl)uronium tetrafluroborate (Fluka)), HOBt (1-hydroxybenzotriazole hydrate (Fluka)) and DIEA (N,N-diisopropylethylamine (Aldrich)) as coupling agents and NMP (N-methylpyrrolidone (HCl Nordic AS) as solvent. Fmoc-deprotection is carried out with piperidine (Fluka).

After assembly of the desired peptide sequence or continuing peptide synthesis according to the LPA-method (Ligand Presenting Assembly; WO 00/18791) peptides were cleaved from the resin with TFA (trifluoroacetic acid (AppliChem)), water and TIS (triisopropylsilane (Aldrich)) and submitted to lyophilisation.

The purity of the peptide was checked with HPLC (high performance liquid chromatography) and MS (electrospray mass spektrometry).

To detect the specificity of antibodies ELISA antigens were synthesized of Aβ₃₃₋₄₂ and Aβ₃₃₋₄₄. The antigens were constructed with a known linear epitope of 8 amino acids for a mouse monoclonal antibody (MAb 35.2 [Birkelund et al. 1994]) and coupled to BSA (bovine serum albumine (Sigma)) through N-terminal (or C-terminal as desired) added cysteine by means of SPDS (N-succinimidyl 3-(2-pyridyidithio)propionate (Sigma)) as bifunctional linker using standard procedure. The epitope was included to make an internal control for immunoglobulin quantification in the ELISA:

BSA-Cystein-(Epitope for MAb)-Aβ-Sequence

BSA-C-₃₆₀NKGVN PDE₃₆₇-₃₃GLMVGGVVIA₄₂
BSA-C₃₆₀NKGVN PDE₃₆₇-₃₃GLMVGGVVIATV₄₄

LPA Technology

The amyloid peptide sequence was assembled as described and further two lysines were coupled to the sequence for hydrophilicity enhancement. After N-terminal deprotection ½ equivalent of Fmoc-imino diacetic acid (Fluka) were coupled to the amyloid resin followed by cyclization between the remaining free amino and carboxy groups. After imino group Fmoc-deprotection the desired LPA was cleaved from the resin with TFA, water and TIS as described above. The purity of the peptide was checked with HPLC and MS.

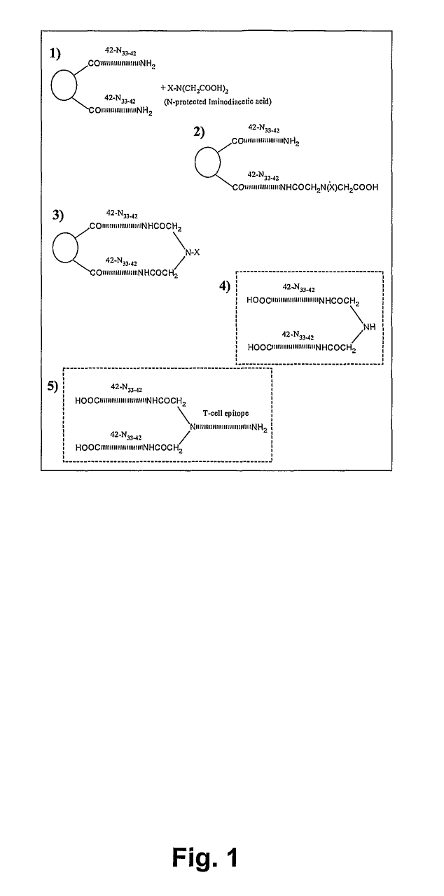

For an illustrative description of the generation of dimeric peptides by using the LPA technology, please see FIG. 1.

Synthesis of T Cell Epitopes

Alternatively the Fmoc-βAla-OH is coupled to imino group after Fmoc-deprotection (see above LPA technology) to give Fmoc-βAla-LPA-amyloid-peptide resin for continued T-cell epitope synthesis. The T-helper cell Tetanus epitope was assembled on all the Fmoc-βAla-LPA-amyloid peptide-resins followed by Fmoc-deprotection and cleavage from the resin as described above.

The purity of the peptide was checked with HPLC and MS.

The human T-helper cell epitope of tetanus toxin that was used in the present invention has previously been shown by the inventors to be immunogenic in mice. The eptiope p30 is 21 amino acids long and localized at amino acid 947-967 in the tetanus toxin. This epitope was described to have promiscuous binding to human MHC class II molecules and it is also recognized by T cells, i.e. the epitope should be immunogenic in both humans and mice. To increase the likelihood that the epitope is recognized in mice it was extended with 4 amino acids in both the N-terminal and C-terminal end and was named p30ex. To confirm that the epitope functioned in mice, three groups of C57/black mice were respectively immunized with 25 μg p30ex in Alhydrogel, with 50 μl Tetanus vaccine (positive control)(SSI, Denmark) and with 50 μl 0.1% Alhydrogel (negative control). The vaccination was performed by intramuscular injection 3 times with 1-week.

Three weeks later spleen cells were harvested and subsequently stimulated with p30ex and tetanus toxoid as described below.

Proliferative Assay

Spleen cells were obtained by rupture the spleen in a homogenizer in RPMI 1640 medium with 10% FCS. The cells were counted and diluted to 10⁶ cell/ml. 200 μl (200.000 cells) were added to each well, triple determination as performed. Antigen was added (1-0.001 μg/ml final concentration). The cells were incubated at 37° C., 5% CO₂ and 85% humidity. After 2 days 1 μCi [³H]-thymidin was add per well. After 18 hours the cells were harvested with a Tomtec device and the incorporation of [³H]-thymidin was measured in a Wallac trilux 1450 microbeta counter.

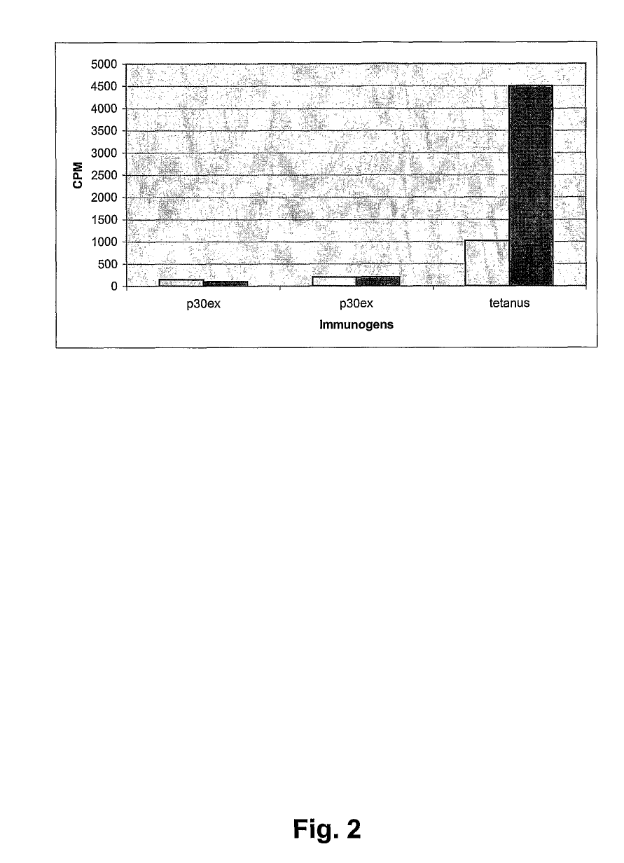

By itself p30ex was not able to provoke a significant immune response, but when the mice were immunized with tetanus toxoid a good response was observed (see FIG. 2).

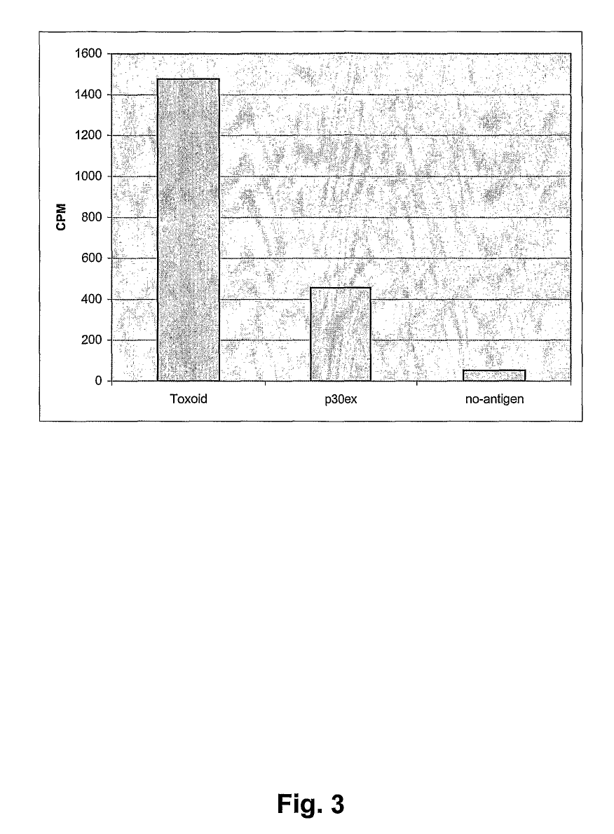

To determine whether p30ex also functioned in humans peripheral blood mononucleated cells (PBMCs) were obtained from an individual immunized with tetanus vaccine (SSI) 3 months earlier. The tetanus toxoid gave a good proliferative response and also p30ex gave a response, similar to what was seen in the tetanus-vaccinated mouse (see FIG. 3). The experimental conditions were identical to the mice experiment except PBMC were used instead of spleen cells, as described above.

Due to these findings it was decided to pre-stimulate mice with tetanus toxoid 10 days before they were given the antigen constructs with the p30ex epitope, in order to increase the chance that the p30ex epitope could give substantial T-cell help to the antibody production.

### Example 2

### Immunogenicity of Antigen Constructs

The antigen constructs were diluted to 1 mg/ml and 0.2% Alhydrogel was added stepwise so the final concentration was 0.5 mg/ml antigen and 0.1% Alhydrogel (according to Brenntag Product insert). Sixty 10 weeks old C57/balck mice were vaccinated with 50 μl Tetanus vaccine (SSI, Denmark) intramuscularly in the quadriceps muscle in order to develop a T-helper cell response to the p30ex epitope. The mice were in groups of 10. At day 10, 20 and 30 each group was vaccinated with 50 μl intramuscularly. At day 50 serum samples were taken by eye puncture.

ELISA

An ELISA assay with the peptide Aβ₃₃₋₄₂ was used to measure the antibody response elicited by the different antigen constructs. The Aβ₃₃₋₄₂ peptide was used as target in the ELISA assay because it covered all antigens used in the immunization groups, thus making it possible to compare the results of the different immunisation groups directly.

The antigen for the ELISA was synthesized with an N-terminal cysteine followed of 8 amino acids constituting an epitope to a mouse monoclonal antibody (MAb 35.2) (Birkelund et al. 1994) and finally the Aβ₃₃₋₄₂ peptide was added C-terminal. At the N-terminal cysteine the peptide was conjugated to bovine serum albumin (BSA). Antigens were diluted in PBS to a final concentration of 4 μg/ml, and 60 μl per well was added to maxysorb plates (Nunc, Denmark). The plates were incubated at 4° C. for 20 hrs. The plates were emptied and 75 μl of 15% (vol/vol) foetal calf serum (FCS) diluted in PBS was added. The plates were incubated for 1 hour at 37° C. and thereafter the plates were washed in PBS with 0.05% Tween-20 three times. Mouse antibodies were diluted 1:50 in antibody buffer (15% FCS, 0.05% Tween-20 in PBS).

The antibodies were added to the plates in duplicates, as positive control MAb 35.2 was used and as negative control the serum from group Alhydrogel used. The plates were incubated for 1 hour at 37° C., and then washed 3 times. As secondary antibodies, Goat anti mouse IgG HRP conjugated (BioRad, Ca.) diluted 1:4,000 in antibody buffer was used. The trays were incubated for 1 hour at 37° C. The plates were washed 3 times and 50 μl TMB (KemEnTec, Denmark) was added. After 30 minutes incubation at 37° C. the reaction was stopped with 100 μl 1M HCl. The plates were read on a Sunrise (Tecan, Austria) instrument at 450 nm with a reference of 620 nm.

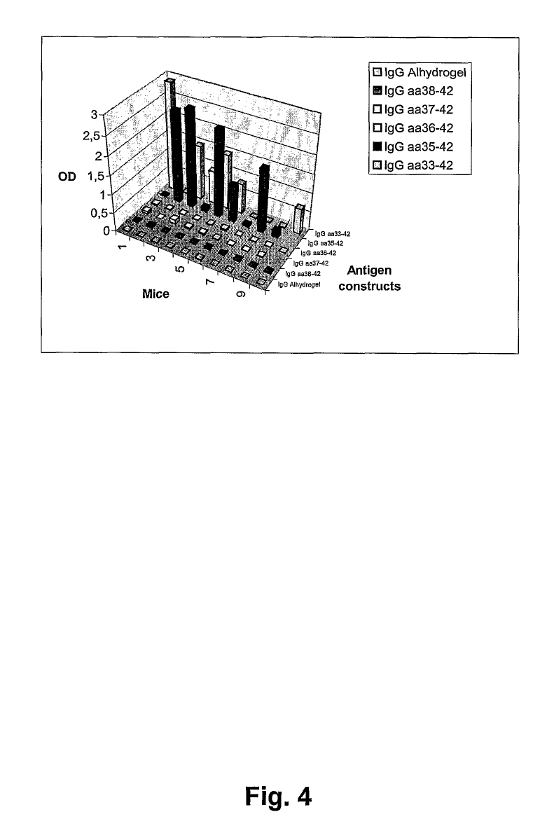

FIG. 4 summarise all the obtained immune responses. Briefly, with P30ex:LPA-KK:(³³GLMVGGVVIA⁴²)₂ 6 out of 10 mice had a good antibody response. Mice immunized with P30ex:LPA-KK:(³⁵MVGGVVIA⁴²)₂ also showed a good response in 6 out of 9 mice. But antigen constructs containing Aβ_{36/37/38-42}, showed no significant reaction over the Alhydrogel group.

In summary, the inventors have shown that peptides of Aβ₁₋₄₂ linked to the p30ex peptide can stimulate the production of antibodies recognizing the C-terminal segment of Aβ₁₋₄₂. However, only the longest peptides, aa 33-42 and aa 35-42, elicited a humoral immune response using the immunization strategy described above. The antigen constructs containing Aβ_{36/37/38-42} elicited no significant antibody response towards Aβ₃₃₋₄₂.

### Example 3

### Generation of Immune Responses to Aβ_{36/37/38-42}

To investigate whether antibody responses could be generated by the smaller Aβ_{36/37/38-42} fragments, the immunogenicity was increased by using a more complex carrier protein than P30ex as well as a more potent adjuvant. The inventors decided to focus on the Aβ₃₇₋₄₂ fragment, and in this new set up, the LPA-KK-(₃₇GGVVIA₄₂)₂ was conjugated to keyhole limpet hemocyanin (KLH)(Sigma-Aldrich, USA) as described below (Aslam, M and Dent A), which is known to give strong T-helper cell response in both mice and rabbits. Furthermore Freunds (Difco, USA) incomplete adjuvant was used.

LPA Aβ_{36/137/138-42} Antigen Preparation

Adjuvants used: Alhydrogel (Brenntag, Denmark), Freunds incomplete, and QS21 (Cambridge Biotech, USA)

The antigen with Alhydrogel was prepared by mixing 400 μl of peptide (1 mg/ml) with 50 μl 0.2% Alhydrogel, after 1 minute and 2 minutes 50 μl additional 0.2% Alhydrogel was added, after 3 minutes 100 μl and after 4 minutes 150 μl. The final composition of the vaccine was 0.5 mg/ml peptide and 1 mg/ml Alhydrogel.

The antigens in Freunds incomplete adjuvant were prepared by mixing 400 μl (1 mg/ml) peptide coupled protein (KLH, tetanus toxoid) with 400 μl Freunds incomplete adjuvant.

The following constructs were used:

Tetanus Toxoid p30ex-βAlanin-LPA-KK-amyloid 33-42:

H-YNDMFNNFTVSFWLRVPKVSASHLEQYGT-βA-N(CH₂COKKGLMVGG
VVIA-OH)₂

Tetanus Toxoid p30ex-βAlanin-LPA-KK-amyloid 35-42:

H-YNDMFNNFTVSFWLRVPKVSASHLEQYGT-βA-N(CH₂COKKMVGGVV
IA-OH)₂

Tetanus Toxoid p30ex-βAlanin-LPA-KK-amyloid 36-42:

H-YNDMFNNFTVSFWLRVPKVSASHLEQYGT-βA-N(CH₂COKKVGGVVI
A-OH)₂

Tetanus Toxoid p30ex-βAlanin-LPA-KK-amyloid 37-42:

H-YNDMFNNFTVSFWLRVPKVSASHLEQYGT-βA-N(CH₂COKKGGVVI
A-OH)₂

Tetanus Toxoid p30ex-βAlanin-LPA-KK-amyloid 38-42:

H-YNDMFNNFTVSFWLRVPKVSASHLEQYGT-βA-N(CH₂COKKGVVI
A-OH)₂

KLH-C-LPA-KK-amyloid 37-42:

Keyhole limpet hemocyanine-C-βAlanin-N(CH₂COKKGGVV
IA-OH)₂

Tetanus Toxoid-C-LPA-KK-amyloid 37-42:

Tetanus Toxoid-C-βAlanin-N(CH₂COKKGGVVIA-OH)₂

For KLH- and Tetanus Toxoid-constructs βAlanin-LPA-KK-amyloid sequences were produced as described above. But instead of the p30ex sequence one cystein was coupled to βAlanin. The peptide was split from the resin, purified on HPLC and lyophilized. The mass was verified by MS. The cystein containing peptide was coupled to KLH or Tetanus Toxoid (SSI) which were activated with SPDP (N-succinimidyl-3-(2-pyridyl dithio) propionate) (Sigma-Aldrich). The coupling efficiency was determined. Lyophilized Aβ₁₋₄₂ was dissolved in H₂O to give a final concentration of 2 mg/ml. The solution was incubated over night at 4° C. for polymerization of Aβ₁₋₄₂. The vaccine was mixed of: 640 μl Aβ₁₋₄₂, 65 μl 10×PBS and 160 μl QS21 (1 mg/ml) according to Cribbs et al. (2003). The antigen was labeled NSH. Control antigen, labelled NSQ: 640 μl H₂O, 65 μl 10×PBS and 160 μl QS21 (1 mg/ml).

Vaccination Protocol

Sixty 10 weeks old C57/balck mice for the antigen groups NSA-E (Table I) and a group of negative controls (named NSØ) were vaccinated with 50 μl Tetanus vaccine (SSI, Denmark) intramuscularly in the quadriceps muscle in order to develop a T-helper cell response to the P30ex epitope. The mice were in groups of 10. At day 10, 20 and 30 each group was vaccinated intramuscularly with 50 μl of one of the vaccines described above. At day 50 serum samples were taken. Ten mice were vaccinated with NSG at day 0, 10, and 20 and serum samples were taken at day 45.

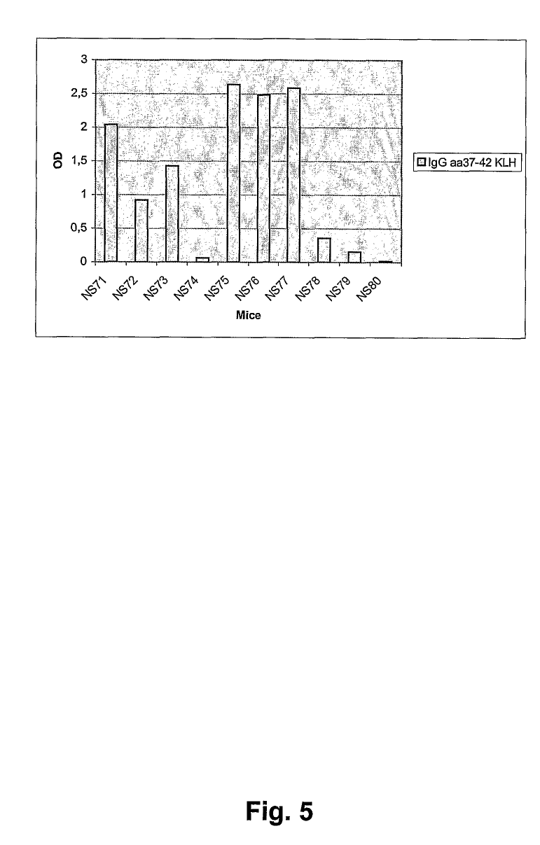

By this immunization method 7 out of 10 mice had significant responses (see FIG. 5). This indicates that lack of response from mice immunized with P30ex:LPA-KK:(³⁶VGGVVIA⁴²)₂, P30ex:LPA-KK:(³⁷GGVVIA⁴²)₂, P30ex:LPA-KK:(³⁸GVVIA⁴²)₂ was not due to lack of B-cells with the right specificity in the C57/black mice, but that only few B-cells with that specificity was present and therefore a strong T-helper cell response provided by KLH using Freund adjuvant was necessary.

### Example 4

### Characterisation of the Direction of the T-Helper Response to Aβ_{33/35/37-42}

The T-helper cells can secrete different interleukins. Th1 cells secret γ-interferon, which stimulates CD8+ cells and results in a humoral response of IgG₂ₐ subclass in mice. The Fc part of IgG₂ₐ is complement activating. The Th1 response is typical for virus infections and infections with intracellular bacteria. Th2 cells secret IL-10 and Transforming Growth Factor-β(TGF-β), which stimulates the humoral immune system to produce IgG₁ subclass antibodies. lgG₁ is not complement activating in mice and only modestly in humans. For a safer Alzheimer vaccine it is essential that the antibodies are not complement activating since such an immune response can mediate lyses of cells coated by the antibodies.

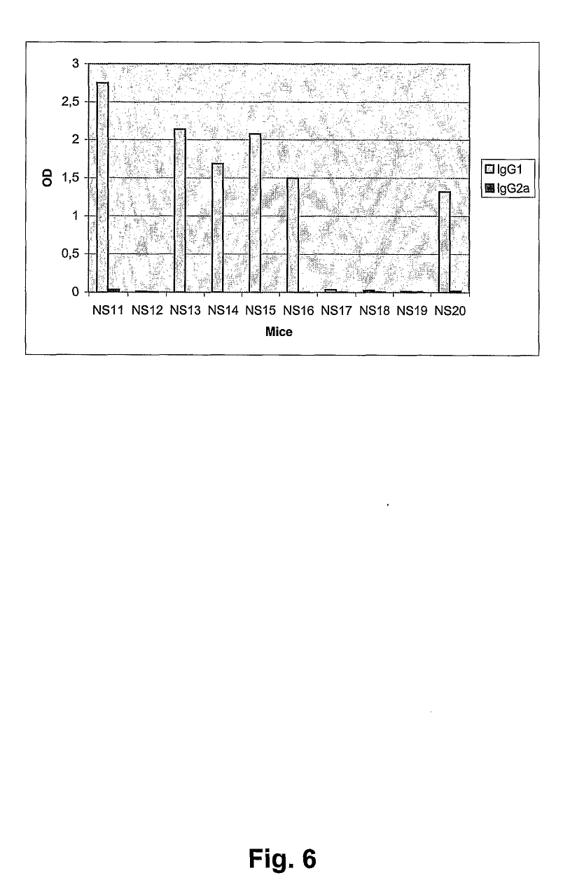

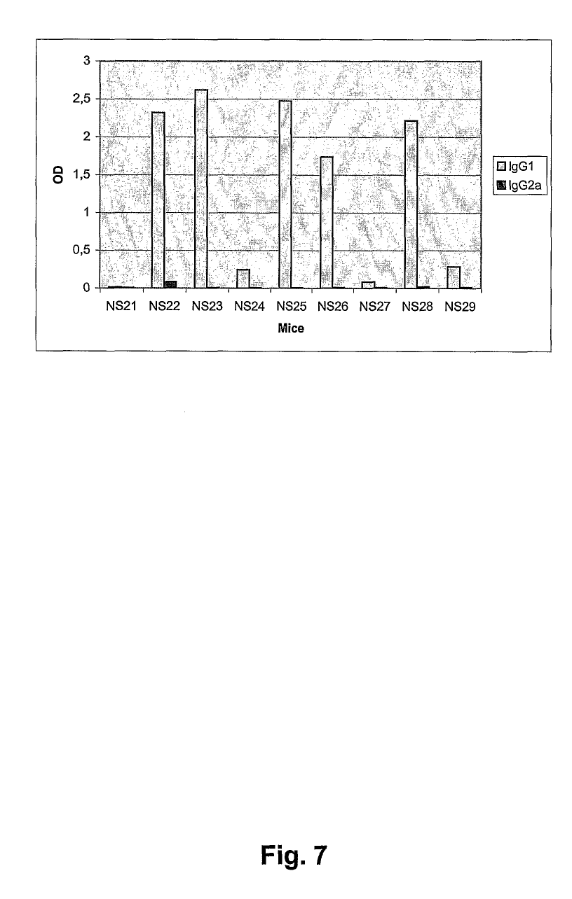

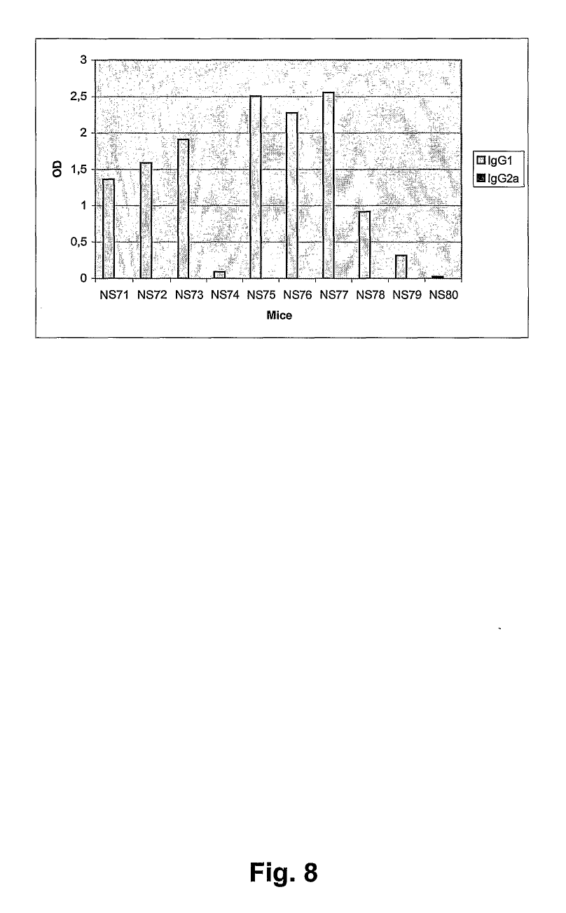

To determine in which direction the antigen constructs had directed the immune response, an ELISA assay with anti mouse IgG₁ and IgG₂ₐ was performed as described in Example 2. However, as secondary antibodies, HRP conjugated goat anti mouse IgG₁ (1:2,000) and IgG₂ₐ (1:4000) from Caltag (USA) were used. The only detectable antibody subclass induced by P30ex:LPA-KK:(³³GLMVGGVVIA⁴²)₂, P30ex:LPA-KK:(³⁵MVGGVVIA⁴²)₂ and KLH:LPA-KK:(³⁷GGVVIA⁴²)₂ was IgG₁ (see FIGS. 6-8). This indicates that the immune response is driven by Th2 cells.

### Example 5

### Specificity of the Immune Response to Aβ_{33/35/37-42}

To determine the specificity of the immune response, ELISA assays were performed as described in Example 2 using two coating antigens: (Aβ₃₃₋₄₂ and Aβ₃₃₋₄₄). Antibodies to an epitope containing the C-terminal group of Aβ₁₋₄₂ would only be positive with the Aβ₃₃₋₄₂ ELISA, whereas the ELISA using Aβ₃₃₋₄₄ would be reduced or completely abolished. Antibodies recognising other epitopes than the C-terminal group of Aβ₁₋₄₂ would be expected to bind in both ELISA assays. Addressing the selectivity directly to Aβ₁₋₄₂ and APP will follow these initial selectivity studies.

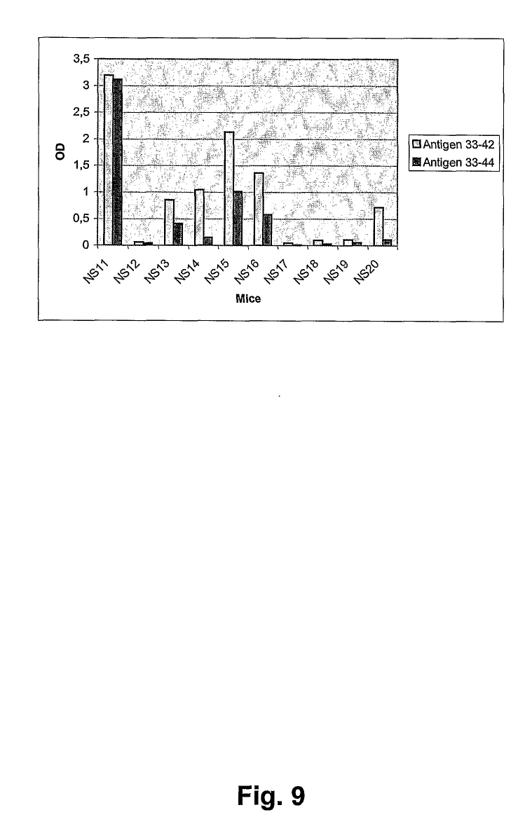

FIG. 9 shows that mice immunized with P30ex:LPA-KK:(³³GLMVGGVVIA⁴²)₂ generated antibodies to both Aβ₃₃₋₄₂ and to Aβ₃₃₋₄₄.

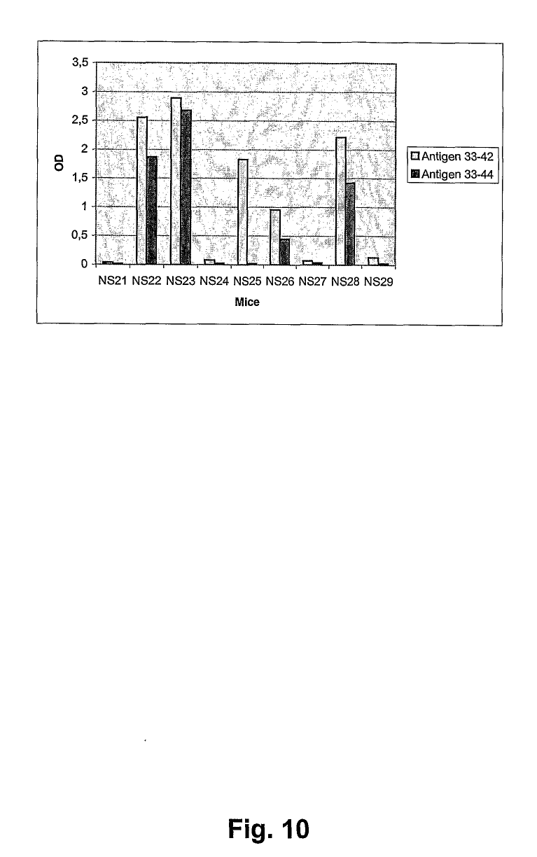

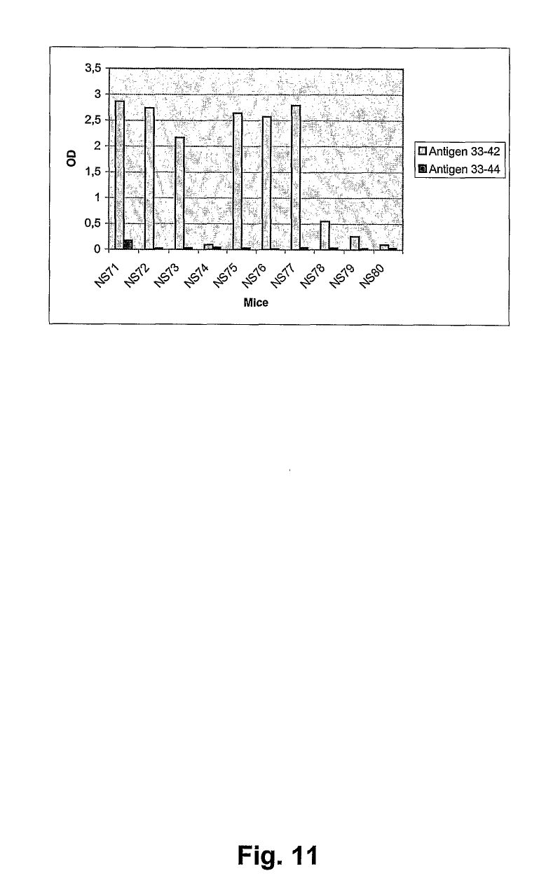

When the mice were immunized with P30ex:LPA-KK:(³⁵MVGGVVIA42)₂ one mouse 25 (NS25) had generated antibodies selectively for the C-terminus of Aβ₁₋₄₂, whereas 4 animals developed antibodies to both Aβ₃₃₋₄₂ and Aβ₃₃₋₄₄, and 4 animals developed weak or no antibody response at all (see FIG. 10). Using the KLH:LPA-KK:(³⁷GGVVIA⁴²)₂ antigen all antibodies were selective for Aβ₃₃₋₄₂ and with no or negligible binding to Aβ₃₃₋₄₄ (see FIG. 11). With the KLH:LPA-KK:(³⁷GGVVIA⁴²)₂ antigen the inventors have been able to generate an immune response in mice that is specifically directed against the C-terminus of Aβ₃₃₋₄₂.

### Example 6

### Study on the T Cell Profile of KLH:LPA-KK:(³⁷GGVVIA⁴²)₂

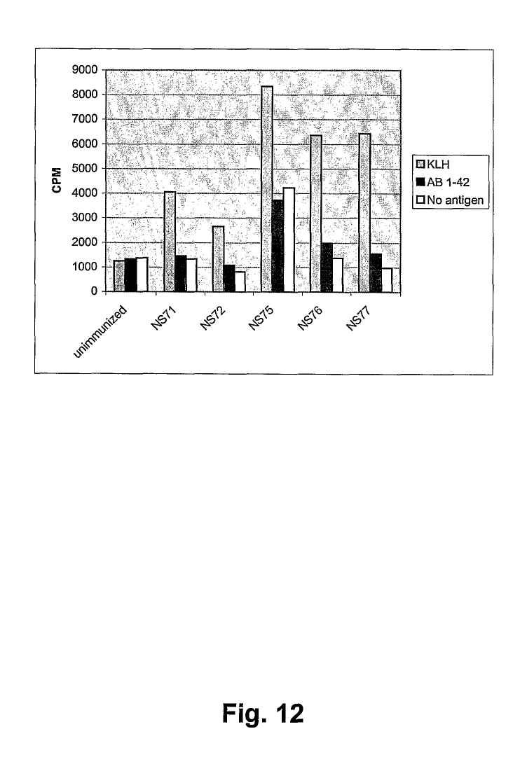

The optimal antigen construct to be used in future clinical studies should most likely not generate a T cell response to Aβ₁₋₄₂, since it could be imagined that a specific T cell response to Aβ₁₋₄₂ potentially could trigger an inflammatory response in the CNS. It was therefore preliminary tested whether the KLH:LPA-KK:(³⁷GGVVIA⁴²)₂ antigen construct gave rise to an Aβ₁₋₄₂ specific T cell response. To do so, 5 mice that responded with antibodies to the KLH:LPA-KK:(³⁷GGVVIA⁴²)₂ construct were given an extra dose of the vaccine 6 weeks after the last immunisation. Four days later spleen cells from these mice were stimulated with Aβ₁₋₄₂ and KLH as positive control as described in Example 1 (see FIG. 12).

There was a good response to KLH whereas the response to Aβ₁₋₄₂ was not significantly greater than background. This indicates that the activated pool of T cells is more primed to react with the T cell epitope rather than Aβ₁₋₄₂. Despite the lack of Aβ₁₋₄₂ specific T cells, there is thus substantial T cell support to generate antibodies specific for Aβ₃₇₋₄₂. This is essential for generation of an effective but safe immune response.

### Example 7

### Design of a New Antigenic Construct Tetanus Toxoid:LPA-KK:(³⁷GGVVIA⁴²)₂

As mentioned above, the construct KLH:LPA-KK:(³⁷GGVVIA⁴²)₂ gave a strong and specific immune response of the Th2 type, whereas the p30ex:LPA-KK:(³⁷ GGVVIA⁴²)₂ was unable to generate a meaningful response in the current immunisation regimen. This indicates that p30ex was insufficient as T cell epitope. In a new round of synthesis, the inventors have therefore focus on replacing p30ex with a sufficient T cell epitope that is also suitable for human use. The initial experiments indicate that full-length tetanus toxoid (TT) could be a safe and potent T cell epitope for human use.

The Following Constructs is Synthesised:

TT:LPA-KK:(³⁷GGVVIA⁴²)₂ and as back-up

TT:LPA-KK:(³⁶VGGVVIA⁴²)₂ and

TT:LPA-KK:(³⁸GVVIA⁴²)₂

The immunogeneticy of TT:LPA-KK:(³⁷GGVVIA⁴²)₂ in different adjuvants (e.g. Freunds, Alhydrogel) is validated. Groups of mice is immunised with:

0.1% Alhydrogel tetanus TT: LPA-(³⁷GGVVIA⁴²)

Freunds adjuvans TT:LPA-KK:(³⁷GGVVIA⁴²)

The immune response raised by TT:LPA-KK:(³⁷GGVVIA⁴²) is analysed by ELISA for binding to the following peptides:

Aβ₁₋₄₀

Aβ₁₋₄₂

Aβ₁₋₃₃

Aβ₃₃₋₄₂

Aβ₃₃₋₄₄

Ratio of immunoglobulin production IgG2a (Th1 indicator) versus IgG1 (Th2 indicator). After immunization mice with TT:LPA-KK:(³⁷GGVVIA⁴²), two methods are used to determine the direction of the T-helper cell respones:

ELISpot assays are prefomed by measuring INFγ (Th1) and IL-4 (Th2) producing T-helper cells after stimulation with TT or Aβ.

Measuring of the ratio of IgG₁ and IgG₂ₐ antibodies to ³⁷GGVVIA⁴² gives an indication of the immune response direction after the immunization. If the ratio is of IgG₁ the T-helper cell response is of the TH2 type.

### Example 8

### Prophylactic Efficacy of Amyloid Beta Against AD

This examples describes administration of the conjugates

| | |
|---|---|
| KLH:LPA-KK: | (³⁷GGVVIA⁴²)₂ |
| TT:LPA-KK: | (³⁷GGVVIA⁴²)₂ |

to transgenic mice overexpressing APP with a mutation at position 717 (PDAPP mice as described by Games et al., Nature) that predisposes them to develop Alzheimer's-like neuropathology. By fifteen months of age, these mice exhibit levels of amyloid beta depositions equivalent to that seen in Alzheimer's disease.

1. Source of Mice

Sixty PDAPP heterogenic female mice is randomly divided into the following groups: 10 mice to be injected with each of the conjugate constructs in two different adjuvants, 5 mice to be injected with PBS/adjuvant or PBS, and 10 uninjected controls.

2. Preparation of Immunogens and Injections

The antigens are prepared for immunisation by mixing 400 μl of conjugates (1 mg/ml) with 50 μl 0.2% Alhydrogel. After 2 minutes, additional 50 μl of 0.2% Alhydrogel is added, after 3 minutes 100 μl and after 4 minutes 150 μl. The final composition of the vaccine is 0.5 mg/ml peptide and 1 mg/ml Alhydrogel.

The antigens in Freunds incomplete adjuvant are prepared by mixing 400 μl (1 mg/ml) of each conjugate with 400 μl Freunds incomplete adjuvant. For each injection, 50 μl of one of the vaccines described above are given intramuscularly at day 0, followed by boosts of the same amount of immunogen in either Alhydrogel or Incomplete Freund's adjuvant (IFA) at 2 weeks and 4 weeks. Five additional doses in the respective adjuvants are given at monthly intervals. PBS injections follows the same schedule and mice are injected with a 1:1 mix of PBS/Adjuvant at 50 μl per mouse, or 50 μl of PBS per mouse.

3. Titration of Mouse Bleeds, Tissue Preparation and Immunohistochemistry

The immunised mice are bleed and the titers of the mice specific to the C-terminal part of amyloid beta 1-42 is monitored every other month using an ELISA method as described in the previous examples. Mice are sacrificed at 13 months and subdued to histological and immunohistochemical examinations to compare the progression of Alzheimer-like pathology in the animals. The histological and immunohistochemical examinations are performed according to general describtions of materials and methods.

The examinations include a description of the pattern and quantification of amyloid deposits in the brain, in particular in the hippocampus, as well as in the frontal and cingulate cortices. This is done using amyloid beta specific antibodies, e.g the monoclonal antibody (mAb) 3D6. The deposition of neuritic plaques in the transgenic mice is typically visualized with the human APP antibody 8E5.

The level of astrocytosis, which is characteristic of plaque-associated inflammation, is measured by quantifying the number of GFAP-positive astrocytes using standard immunohistochemical methodology. The GFAP-positive astrocytes can further be counter-stained with Thioflavin S to detect co-localization with amyloid beta deposits.

Sections of the mouse brains are also reacted with a monoclonal antibody specific for MAC-1, a cell surface protein. MAC-1 (CD11b) is an integrin family member and exists as a heterodimer with CD18. The CD11b/CD18 complex is present on monocytes, macrophages, neutrophils and natural killer cells (Mak and Simard). The resident MAC-1-reactive cell type in the brain is likely to be microglia based on similar phenotypic morphology in MAC-1 immunoreacted sections. Plaque-associated MAC-1 labeling will be lower in the brains of mice treated with an effective vaccine as compared to the PBS control group due to the lack of an Amyloid beta-induced inflammatory response.

The above analysis will show that no or very little amyloid is deposited in the brains of mice, which have received one of the active immunogens. The accompanying pathological consequences, such as gliosis and neuritic pathology, will also be significantly reduced or absent. The vaccinated mice will show essentially the same lack of pathology as control nontransgenic mice.

### Example 9

### Passive Vaccination Strategy

Mice are immunized with the LPA complex conjugated with an immunogenic carrier protein (ex KLH, tetanus toxoid or others) and a peptide encompassing the C-terminal part (33-42) of Aβ1-42. The C-terminal part is here defined as the stretch of amino acid starting with number 33 to 37 ending with amino acid position 42 of Aβ1-42, preferable Aβ37-42.

After several rounds of immunizations spleen cells are isolated from immunized mice. Splenic B cells are fused with a suitable immortalized B cell fusion cell line and B cell hybridomas are generated. The generated B cell hybridomas are culture as pooled clones in micro titer plates and screened for selectivity the C-terminal part of Aβ1-42. Selective pools of B cell hybridomas are single cell cloned and following analyzed for the selectivity of the C-terminal part of Aβ1-42.

The selectivity profile of B cell hybridoma clones selective for the C-terninal part of Aβ1-42 are analyzed in vitro against 1-40, 1-42, 1-33, 33-42, 37-42 and 33-44, Aβ fibrils, Aβ plaques and the amyloid precursor protein using ELISA and immune histology.

Monoclonal antibodies are tested in different strains of mice: systemic and CNS changes in pro-inflammatory cytokines are measured and histological CNS changes are measured.

The efficacy profile of an antibody specific for the C-terminal part of Aβ1-42 are assessed in vitro by measuring resolution of amyloid plaques and in vivo in murine transgenic disease models of Alzheimer disease, measuring cognitive improvements and reduced amyloid load in the CNS.

The optimal efficacious antibody is humanised for clinical trials (either by molecular engineering of B cell hybridomas from wild-type mice or constructing new hybridomas using transgenic mouse models with “human-like” immune system).

### Example 10

### Production of Nonhuman and Murine Humanized Antibodies

The production of antibodies, e.g., humanized murine, murine, guinea pig, primate, rabbit or rat, can be accomplished by, for example, immunizing the animal with the conjugate Tetanus toxoid:LPA-KK:(³⁷GGVVIA⁴²)₂. A larger conjugate comprising the carboxyl terminus of amyloid beta or an immunogenic fragment comprising the carboxyl terminus of amyloid beta(1-40) or (1-43) can also be used. The conjugate can be synthesized as described in the previous examples. Optionally, the immunogen can be administered with an adjuvant. Several types of adjuvant can be used as described below. Complete Freund's adjuvant followed by incomplete adjuvant is preferred for immunization of laboratory animals. Rabbits or guinea pigs are typically used for making polyclonal non-human antibodies. Mice are typically used for making monoclonal non-human antibodies. Humanized mice are typically used for making humanized monoclonal antibodies. Antibodies are screened for specific binding to the carboxyl terminus of the relevant amyloid beta peptide as used in the antigenic conjugate, e,g. amyloid beta(1-42). Optionally, antibodies are further screened for non-binding to other regions of amyloid beta or other proteins in general. The screening can be accomplished, for example, by Western blot or ELISA. Alternatively, epitope specificity can be determined by a competition assay is which a test and reference antibody compete for binding to the carboxyl terminus of amyloid beta. If the test and reference antibodies compete, then they bind to the same epitope or epitopes sufficiently proximal that binding of one antibody interferes with binding of the other. The preferred isotype for such antibodies is mouse isotype IgG2a or equivalent isotype in other species. Mouse isotype IgG2a is the equivalent of human isotype IgG1.

### REFERENCES

Aslam, M and Dent A. Bioconjugation, Protein Coupling Techniques for the Biomedical Sciences, Macmill\dot{a}n Reference Ltd. (1998).
Bard, F. et al. Nature Med. (2000) 6, 916.
Birkelund S et al. Infect Immun. 62:2051-2057 (1994).
Cribbs D H, Int Immunol. 15:505-14 (2003).
Decision Resources (in the report “Outlook for Neurodegenerative Disease Therapy Markets”)
Gong, Y et al. Proc. Natl Acad. Sci. USA (2003), 100, 18, 10417-10422
Hock et al., Neuron 38, 547 (2003)
Morgan et al., Nature (2000), 408: 982-985
Mucke, L et al. J. Neurosci. 20, 4050-4058 (2000)
Neuberger M S, Eur J Immunol. 11:1012-6 (1981).
Panina-Bordignon P, Eur J Immunol. 19:2237-42 (1989).
Schenk, D. et al. Nature 400 (1999), 173
Walsh, D M et al. Nature (2002), 416, 535-539;
Weiner, H L et al. Ann Neurol. 2000, 48, 567-579

1. A conjugate having the following structure 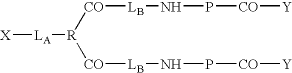
wherein
R represents —N(CH₂—)₂, —NHCH<; or —NHCH(CH₂—)₂,
X represents a hydrogen or a peptidic group, and
L_{A} is optionally present and is an amino acid or a peptide containing at least 2 amino acid residues,
L_{B} is optionally present and is an amino acid or a peptide containing at least 2 amino acid residues,
P is a peptide selected from full length or fragments of amyloid proteins or proteins with substantial similarity to an amyloid protein,
Y is OH or NH₂,
and pharmaceutically acceptable salts thereof.

2. A conjugate according to claim 1, which upon administration to a mammal is capable of eliciting a production of antibodies having specificity towards the conjugate itself, and inducing an immune response in the mammal, thereby preventing or reducing amyloid-induced cellular toxicity and/or the formation of amyloid fibrils, plaques and/or deposits.

3. A conjugate according to claim 2, wherein the antibodies produced are having specificity towards one or more C-terminally presented P peptides of the conjugate.

4. A conjugate according to claim 1, wherein P is a fragment comprising at least one region of an amyloid protein.

5. A conjugate according to claim 4, wherein the region is selected from the group comprising the C-terminal region, beta sheet region, cytotoxic region, GAG-binding site region, or macrophage adherence region.

6. A conjugate according to claim 1, wherein the amyloid proteins are derived from amyloid precursor proteins selected from the group comprising serum amyloid A protein (ApoSSA), immunoglobulin light chain, immunoglobulin heavy chain, ApoA1, transthyretin, lysozyme, fibrinogen alpha chain, gelsolin, cystatin C, amyloid beta protein precursor (beta.-APP), betas microglobulin, prion precursor protein (PrP), atrial natriuretic factor, keratin, islet amyloid polypeptide and synuclein or any polypeptides with substantial similarity to any of the above.

7. A conjugate according to claim 1, wherein the amyloid proteins are selected from amyloid beta (1-43), amyloid beta (1-42), amyloid beta (1-41), amyloid beta (1-40), amyloid beta (1-39) and amyloid beta (1-38).

8. A conjugate according to claim 7, wherein P is a fragment of amyloid beta (1-43), amyloid beta (1-42), amyloid beta (1-41), amyloid beta (1-40), amyloid beta (1-39) or amyloid beta (1-38).

9. A conjugate according to claim 8, wherein P contains the C-terminus of amyloid beta.

10. A conjugate according to claim 8, wherein P is a fragment of 10 amino acids from the C-terminus of amyloid beta.

11. A conjugate according to claim 8, wherein P is a fragment of 9 amino acids from the C-terminus of amyloid beta.

12. A conjugate according to claim 8, wherein P is a fragment of 8 amino acids from the C-terminus of amyloid beta.

13. A conjugate according to claim 8, wherein P is a fragment of 7 amino acids from the C-terminus of amyloid beta.

14. A conjugate according to claim 8, wherein P is a fragment of 6 amino acids from the C-terminus of amyloid beta.

15. A conjugate according to claim 8, wherein P is a fragment of 5 amino acids from the C-terminus of amyloid beta.

16. A conjugate according to claim 8, wherein P is a fragment of 4 amino acids from the C-terminus of amyloid beta.

17. A conjugate according to claim 8, wherein P is a fragment of 3 amino acids from the C-terminus of amyloid beta.

18. A conjugate according to claim 12, wherein P is fragment 35-42 of amyloid beta (1-42).

19. A conjugate according to claim 13, wherein P is fragment 36-42 of amyloid beta (1-42).

20. A conjugate according to claim 14, wherein P is fragment 37-42 of amyloid beta (1-42).

21. A conjugate according to claim 15, wherein P is fragment 38-42 of amyloid beta (1-42).

22. A conjugate according to claim 16, wherein P is fragment 39-42 of amyloid beta (1-42).

23. A conjugate according to claim 17, wherein P is fragment 40-42 of amyloid beta (1-42).

24. A conjugate according to claim 1, wherein X is a T cell epitope.

25. A conjugate according to claim 24, wherein X is a human T cell epitope including full-length tetanus toxoid, tetanus toxoid fragment FNNFTVSFWLRVPKVSASHLE and tetanus toxoid fragment YNDMFNNFTVSFWLRVPKVSASHLEQYGT, or a rodent T cell epitope including QYIKANSKFIGITEL.

26. A conjugate according to claim 1, wherein X is Keyhole Limpet Hemocyanin or BSA.

27. A method for the treatment, amelioration and/or prophylaxis of an amyloid-related disease in a mammal, the method comprising administering to the mammal an antigenic amount of a conjugate as recited in claim 1, wherein the conjugate elicits the production of antibodies having specificity towards the conjugate itself and induces an immune response in the mammal, thereby preventing or reducing amyloid-induced cellular toxicity and/or the formation of fibrils, plaques and/or amyloid deposits.

28. A method according to claim 27, wherein the antibodies produced are specific towards one or more C-terminally presented P peptides of the conjugate.

29. A method according to claim 27, the method further comprises the administration of an adjuvant together with the conjugate.

30. A method according to claim 29, wherein the adjuvant is selected from the group comprising complete Freunds adjuvant, incomplete Freunds adjuvant, QS21, Aluminium hydroxide gel, MF59 and calcium phosphate.

31. A method according to claim 27, wherein the amyloid-related disease is Alzheimer's disease, Down's syndrome, vascular dementia or cognitive impairment.

32. Use of a conjugate as defined in claim 1 for the preparation of a pharmaceutical composition for the treatment and/or prophylaxis of an amyloid-related disease in a mammal.

33. A vaccine comprising a conjugate as recited in claim 1 together with an adjuvant.

34. A vaccine according to claim 33, wherein the adjuvant is selected from the group comprising complete Freunds adjuvant, incomplete Freunds adjuvant, QS21, Aluminium hydroxide gel, MF59 and calcium phosphate.

35. A method for producing an antibody in a mammal, the method comprising administering to the mammal an antigenic amount of a conjugate as recited in claim 1, wherein the conjugate elicits the production of antibodies having specificity towards the conjugate itself.

36. A method according to claim 35, wherein antibodies produced are specific towards one or more C-terminally presented P peptides of the conjugate.

37. A method according to claim 35, which further comprises the step of generating hybridoma cells by somatic cell hybridization for the production of monoclonal or polyclonal antibodies.

38. A method according to claim 35, wherein the mammal is a mouse or humanized mouse.

39. An antibody having specificity towards a conjugate as recited in claim 1.

40. An antibody as recited in claim 39, wherein the antibody is specific towards one or more C-terminally presented P peptides in the conjugate.

41. An antibody according to claim 39, which is monoclonal.

42. An antibody according to claim 39, which is humanized or chimeric.

43. An antibody according to claim 39, which is produced administering to a mammal an antigenic amount of the conjugate.

44. A method for the treatment and/or prophylaxis of an amyloid-related disease in a mammal, the method comprising administering to the mammal an antibody as recited in claim 39, thereby preventing or reducing amyloid-induced cellular toxicity and/or the formation of fibrils, plaques and/or amyloid deposits.

45. A method according to claim 44, wherein the amyloid-related disease is Alzheimer's disease, Down's syndrome, vascular dementia or cognitive impairment.


SEQUENCE LISTING (9 sequences)

SEQ ID NO: 1 - protein, 42 aa, Homo sapiens [sequence omitted]

SEQ ID NO: 2 - protein, 10 aa, Homo sapiens
GLMVGGVVIA

SEQ ID NO: 3 - protein, 8 aa, Homo sapiens
MVGGVVIA

SEQ ID NO: 4 - protein, 7 aa, Homo sapiens
VGGVVIA

SEQ ID NO: 5 - protein, 6 aa, Homo sapiens
GGVVIA

SEQ ID NO: 6 - protein, 5 aa, Homo sapiens
GVVIA

SEQ ID NO: 7 - protein, 21 aa, Clostridium tetani
FNNFTVSFWLRVPKVSASHLE

SEQ ID NO: 8 - protein, 29 aa, Clostridium tetani
YNDMFNNFTVSFWLRVPKVSASHLEQYGT

SEQ ID NO: 9 - protein, 15 aa, Clostridium tetani
QYIKANSKFIGITEL